(19) United States
(12) Patent Application Publication
(10) Pub. No.: US 2024/0315972 A1
(43) Pub. Date: Sep. 26, 2024

(54) PROTEIN CONTAINING BIO-ACTIVE COMPOSITIONS COMPRISING CELLULOSE MICROPARTICLE CARRIERS

(71) Applicant: Zidkiyahu Simenhaus, Galil Elyon (IL)

(72) Inventor: Zidkiyahu Simenhaus, Galil Elyon (IL)

(73) Assignee: Zidkiyahu Simenhaus, Galil Elyon (IL)

(21) Appl. No.: 18/574,491

(22) PCT Filed: Jul. 7, 2022
(86) PCT No.: PCT/IL2022/050739

§ 371 Date: Dec. 27, 2023

Related U.S. Application Data
(60) Provisional application No. 63/220,574, filed on Jul. 12, 2021.

(51) Int. Cl. A61K 9/16; A23L 29/262; A23L 33/105; A23L 33/18; A61K 8/73; A61K 9/00; A61K 9/14; A61K 9/20; A61K 31/00; A61K 36/9066; A61K 38/02; A61K 38/28; A61K 38/47; A61K 38/48; A61K 47/38

(52) U.S. Cl. CPC A61K 9/1652; A23L 29/262; A23L 33/105; A23L 33/18; A61K 8/731; A61K 9/0014; A61K 9/0053; A61K 9/146; A61K 9/2054; A61K 9/2077; A61K 31/658; A61K 36/9066; A61K 38/02; A61K 38/28; A61K 38/47; A61K 38/4826; A61K 47/38

(57) ABSTRACT

Compositions comprising ground or milled cotton fibers comprising oxidized cellulose microparticles carriers of between about 10-15.5 pm in diameter and of between about 0.5-100 pm in length, wherein the oxidized cellulose microparticles carriers contain aldehyde functional groups, and wherein the composition further comprises at least one protein or polypeptide or therapeutic compound, bound to the oxidized cellulose microparticles carrier through the aldehyde functional group. The invention also provides for methods of preparing the compositions and methods for improving a release profile of a protein, polypeptide or therapeutic compound in a composition for delivery to a mucosal surface of a subject, the method comprising activating cotton fibers containing-material comprising cellulose via application of a solution containing aldehyde functional groups to yield oxidized cellulose components of the fibers, applying a solution of one protein or polypeptide or therapeutic compound to the activated textile and grinding or milling the activated textile to obtain oxidized cellulose microparticles carriers with protein or polypeptide or therapeutic compound covalently bound thereto and incorporating the ground microparticles within a composition.

### BACKGROUND OF THE INVENTION

Oral delivery of active agents is a particularly desirable route of administration, because of safety and convenience considerations. In addition, oral delivery provides for more accurate dosing than multi-dose vials and can minimize or eliminate the discomfort that often accompanies repeated hypodermic injections.

There are many obstacles to successful oral delivery of protein molecules especially biological macromolecules, e.g., Insulin. More importantly, the active conformation of many biological molecules may be sensitive to a variety of environmental factors, such as temperature, oxidizing agents, pH, freezing, shaking and shear stress. In planning oral delivery systems comprising biological molecules as an active agent for drug development, these complex structural and stability factors must be considered.

In addition, in general, for medical and therapeutic applications, where a biological molecule is being administered to a patient and is expected to perform its natural biological function, delivery vehicles must be able to release active molecules, at a rate that is consistent with the needs of the particular patient or the disease process

It is well known in medical practice to use proteins and other bio-active substances in the form of tablets, capsules, solutions for treating diseases and disorders; and also, dressings for treating inflammatory wounds, burn and stopping bleeding.

In the dietary supplements field tablets, capsules and liquids containing proteins or other bio-active substances as the active component are currently in use for treatment of disorders.

In cosmetics formulations, it is common to use compositions comprising water-in-oil emulsions containing a protein(s) or another bio-active substance(s) as the active ingredient dispersed in an oily phase.

Various proteins are known to possess therapeutic properties, for example, Trypsin, Insulin, Resveratrol, Curcumin have been described as contributing to combat different diseases and disorders.

Trypsin's therapeutic properties may facilitate mitigating Airway infections which are caused by/accompany exercise, colon cancer, rectal cancer, diabetes, infections of the kidney, bladder, or urethra (urinary tract infections or UTIs), multiple sclerosis (MS), muscle soreness caused by exercise, osteoarthritis, skin damage caused by radiation therapy (radiation, dermatitis), sprains, swelling after surgery, wound healing, to name a few.

Trypsin has been shown to be effective against influenza viruses in vitro and on skin. Trypsin is shown to be 3-12 times more effective in degrading large native proteins than its mesophilic analogue. This is in agreement with previous findings where trypsin was found to be the most active among twelve different proteases in cleaving various cytokines and pathological proteins. Furthermore, results show that trypsin has high efficacy against herpes simplex virus type 1 (HSV-1) and the respiratory syncytial virus (RSV) in vitro. The results on the antipathogenic properties of trypsin are important because rhinovirus, RSV, and influenza are the most predominant pathogenic viruses in upper respiratory tract infections.

Trypsin promotes efficient influenza vaccine production in MDCK cells by interfering with the antiviral host response Arnheiter H, Haller O (1983) Mx gene control of interferon action: different kinetics of the antiviral state against influenza virus and vesicular stomatitis virus. J Virol 47(3):626-630 Importance Trypsin is commonly used in Madin-Darby canine kidney (MDCK) cell culture-based influenza vaccine production to facilitate virus infection by proteolytic activation of viral haemagglutinin, which enables multi-cycle replication. In this study, They were able to demonstrate that trypsin also interferes with pathogen defense mechanisms of host cells. In particular, a trypsin concentration of 5 BAEE U/mL (4.5 μg/mL porcine trypsin) used in vaccine manufacturing strongly inhibited interferon (IFN) signaling by proteolytic degradation of secreted IFN.

Another therapeutic protein, Reservatrol, has been described as being protective against heart disease, in its suspected role in reducing inflammation, lowering LDL, and making it more difficult for clots to form that can lead to heart attack. A suspected role in limiting the spread of cancer cells and killing cancer cells has been attributed to Reservatrol, as well. In Alzheimer's disease, Reservatrol has been described as potentially protecting nerve cells from damage and reducing plaque buildup that can lead to the disease. Resveratrol helps prevent insulin resistance, and therefore useful in treating diabetes. Resveratrol has been described as activating the SIRT1 gene, which protects the body against the effects of obesity and the diseases of aging.

Curcumin has been described as possessing anti-inflammatory and anti-oxidant properties which impact Low-level Inflammation, also Heart disease, Cancer, Metabolic syndrome, Alzheimer's and other degenerative condition and oxidative damage, potentially neutralizing Free Radicals.

It is a long-standing goal in the pharmaceutical industry to provide oral compositions of therapeutic proteins. To date these efforts have been confounded poor absorption of proteins in the gastrointestinal tract, enzymatic degradation and other aspects. It would be desirable to create an oral pharmaceutical formulation of a drug such as trypsin, insulin, resveratrol, curcumin etc., which formulation would provide sufficient absorption and pharmacokinetic/pharmacodynamic properties to provide the desired therapeutic effect.

U.S. Pat. No. 5,412,96, Pat. EP 0138544A2 concern a method for overcoming trypsin inhibition by soybean trypsin inhibitor (STI) in soy-based foodstuffs. In the method of the invention, the foodstuff either during its processing, or subsequently, is treated with an effective amount of starfish trypsin-1 (DIT1) and with an effective amount of supplementary proteolytic enzyme, encapsulated so as to protect it from digestion in the stomach, but to permit release in the upper small intestine so as to interact with STI as it progresses through the digestive tract along with the ingested food.

U.S. Pat. No. 4,512,973A is directed to treating soy-based foods with an effective amount of starfish trypsin 1 (DIT1) to inactivate soybean trypsin inhibitor (STI). Also contemplated are the oral administration of starfish trypsin 1 to overcome soybean trypsin inhibitor and pharmaceutical compositions containing starfish trypsin 1.

U.S. Pat. No. 3,324,002 describes anti-inflammatory preparations, to the treatment of human beings for the alleviation of inflammatory conditions, and more particularly to preparations' suitable for systemic anti-inflammatory or mucolytic effect, and to treatment therewith by absorption from the digestive tract, by parenteral administration, and also by rectal or vaginal administration.

US Pat. 20090081184A1 describes methods for maintaining the basal level or reducing the level of cholecystokinin (CCK) in blood plasma of a mammal. Additionally, the methods of this invention are particularly useful for treating abdominal pain in a mammal suffering from acute or chronic pancreatitis and related conditions.

U.S. Pat. No. 3,004,893A describes formulating trypsin or equivalent enzymes in a suitable enteric coated tablet or capsule in which the coating is of such a composition and thickness that it will not break down until the tablet or capsule has reached the ileum or lower part of the small intestine. By this procedure the enzyme is protected so that it gets through the digestive phase in the upper portion of the ileum without entering into digestive action expected of the proteolytic enzymes at this point. Instead, it is not released in the intestine until it is well past the point of the digestive action of the gastric and pancreatic enzymes.

Dietary supplement tablets and capsules in which trypsin or curcumin or resveratrol or any one of others is the active ingredient are also commercially available. These products are declared to be used for treatment of many conditions.

While the promise of protein-based therapeutics has been long described, there is to date insufficient evidence of the efficacy of orally formulated products containing same.

U.S. Pat. No. 7,455,830 discloses oral delivery of insulin in a formulation containing a shell substrate of chitosan, and a core substrate selected from the group consisting of gamma-polyglutamic acid (PGA), alpha-PGA, water soluble salts of PGA and metal salts of PGA;

U.S. Pat. No. 7,470,663 discloses a liquid solution formulated for oral delivery, comprising a substantially monodispersed mixture of conjugates, wherein each conjugate comprises human insulin covalently coupled a carboxylic acid, which is covalently coupled at the end distal to the carboxylic acid moiety to a methyl terminated polyethylene glycol moiety.

U.S. Pat. No. 7,384,914 discloses a method of treating a mammal which has impaired glucose tolerance by administering a therapeutically effective dose of a pharmaceutical formulation comprising insulin and the delivery agent 4-[(2-hydroxy-4-chlorobenzoyl) amino-butanoate (4-CNAB) in an amount which facilitates absorption of the insulin from the gastrointestinal tract of the treated mammal, and

U.S. Pat. No. 6,656,922 which discloses a method for enhancing oral administration of insulin by conjugating insulin to a hydrophobic agent selected from the group consisting of bile acids, sterols, alkanoic acids, and mixtures thereof to result in a hydrophobized macromolecular agent.

US Pat. 20100278922A1 describes a pharmaceutical composition formulated for oral delivery of insulin, comprising a particulate non-covalently associated mixture of pharmacologically inert silica nanoparticles having a hydrophobic surface, a polysaccharide, and insulin suspended in, embedded in or dispersed in an oil or mixture of oils.

Russian Patent No. (11) 2288000(13) C1 describes preparing an orally administered insulin solution containing Insulin at0.005-0.02 mass %, Sodium chloride at 0.5-1.0 mass %, and water up to 100 mass %. Unfortunately, a short-term hypoglycemic effect was evident (see Table No. 1) whereby insulin protein was subjected to the destructive effects of proteolytic enzymes in the stomach.

The ability of a carrier to capture and hold molecules such as enzymes and other protein or any synthetic chemical could be expressed as a ratio between the degree of oxidation of the carrier/excipients to the amount of protein, meaning—The ratio of enzymes—carrier/excipients bonding is determined by the amount of functional groups incorporated in the textile carrier. The efficacy of the protein is determined by the amount of the protein bound to the dialdehyde groups and the releasable activity depends of the nature of the bonding.

The main obstacle in reaching a protein-rich product is the limited nature of binding between the bioactive ingredient and carrier. To overcome this obstacle, earlier and present technologies simply use a higher concentration of bonding groups. These methods, however, comes with significant disadvantages: limited amount of bioactive agent and decreased product activity. For example, GB Patent No. 2,240,040 suggest increasing the oxidation degree, by increasing the conjugates agents i.e., the functional of the product (dressing) from 5% oxidation degree to 25% oxidation degree in order to incorporate large amounts of protein. Subsequently the available releasable amounts of therapeutical agents are being increased from 0.02% covalently bound respectively to 5% oxidation degree to 0.5% covalently bound respectively to 25% oxidation degree.

Referring to the methods above-mentioned, most of them teaching a method to bind proteins by covalent bound. Well known in the art that protein molecules covalently bound seals away significantly portion of the dialdehyde groups. As a result, the availability of binding large amounts of protein is limited. Subsequently, the releasable amount of therapeutical agent is, respectively, limited and the release curb is also slowed, i.e., its activity is degraded.

Due to the well-known method for the calculation of hydrolysis of protein, as per the equation of rate of release:

K[A]″[B[′>[cc . . . [Z]′ . . .

Where K is the rate constant, which is an invariable occurring of a chemical reaction, i.e. how fast the original (starting) substances disappear by hydrolysis.

[A], [B], [C] . . . [Z] . . . , stands for the current existing concentration of the reagent, and

a, b, c, . . . z . . . , are the so-called orders of the reactions.

GB Patent No. 2,240,040 describes a product (dressing) in which the trypsin is bound covalently are as follows: From a dressing at 5% degree of oxidation and respectively 0.02% of protein covalently bound, 50% of the total amount of the protein incorporated is releasable during 36 hours, and in next 36 hours an additional 25% of the initial amount is released, i.e., during 72 hours 75% of the total amount is releasable.

From a dressing of 25% degree of oxidation and respectively 0.5% of protein covalently bound, only 77% of the protein incorporated is releasable. 50% were released during the first 24 hours, and during the next 48 hours 22%-27% were released, i.e., during 72 hours 72%-77% of the initial total amount is releasable.

Despite an enormous body of research there is no ideal means to date of formulating various classes of therapeutic proteins for oral administration.

### SUMMARY OF THE INVENTION

In one embodiment, this invention provides a pharmaceutical drug, the dietary supplements and the cosmetic fields. More particularly the present invention relates to methods for producing a variety of pharmaceutical such tablets and capsules, medicinal and cosmetic creams containing cellulose microparticles carriers, partially oxidized, comprising respectively bioactive substances' molecules and having releasable therapeutic activity.

The present invention relates to the pharmaceutical, the dietary supplements and the cosmetic fields. More particularly the present invention relates to bio-drug tablets and capsules, bio-dietary supplements tablets and capsules orally administered; and to medicinal and cosmetic creams and serums containing cellulose (cotton or viscose) microparticles carriers, partially oxidized, comprising respectively bioactive substances' molecules of proteins and/or combination of proteins and synthetic chemicals having releasable therapeutic activity. And methods for producing the variety of the bio-tablets and capsules, bio-creams and the bio-serums mentioned above.

The trypsin and/or insulin which the present invention displaying herein are illustrative examples of protein. It should be noted that the process of binding for example insulin (a peptide hormone) or trypsin (enzyme) within cellulosic carrier materials and having it in a dry state is, in general, similar to the process of capturing any other enzymes, other proteins, or other bio-active substance, in cellulosic carrier materials.

In some embodiments, this invention provides a composition comprising:
ground or milled cotton fibers comprising oxidized cellulose microparticles carriers of between about 10-15.5 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group.

In some embodiments, the ground or milled cotton fibers comprise oxidized cellulose microparticles carriers.

According to this aspect, and in some embodiments, the oxidized cellulose microparticles carriers are of between about 10-15.5 μm in diameter and of between about 0.5-100 μm in length.

Still according to this aspect, in some embodiments, the oxidized cellulose microparticles carriers contain between about 0.208% w/w. to 12.0% w/w, between 0.026 mg (equiv) to 1.5 mg (equiv), respectively, of aldehyde functional groups per gram of carrier.

Still according to this aspect of the invention and in some embodiments, the carrier may range from about 100 mg to about 500 mg in weight.

In some embodiments, the oxidized cellulose microparticles carriers are bound to at least one protein or polypeptide or therapeutic compound through the aldehyde functional group of the oxidized cellulose microparticles carriers.

In some embodiments, the composition is for oral administration, which in some embodiments is a tablet, capsule, powder, food product, syrup or liquid.

In some embodiments, the at least one protein or polypeptide or therapeutic compound comprises trypsin, insulin, curcumin or reservatrol, or in some embodiments, the at least one protein or polypeptide or therapeutic compound comprises a cannabinoid, or in some embodiments, the at least one protein or polypeptide or therapeutic compound comprises a superoxide dismutase, or in some embodiments, the at least one protein or polypeptide or therapeutic compound comprises glatiramer acetate (copaxsone), or in some embodiments, the at least one protein or polypeptide or therapeutic compound comprises trypsin, reservatrol, curcumin, insulin, lysozyme, or a combination thereof.

In some embodiments, the composition is for topical administration, which in some embodiments, is in the form of a cream, mousse, serum, shampoo, conditioner or makeup.

In some embodiments, the at least one protein or polypeptide or therapeutic compound comprises trypsin, insulin, curcumin or reservatrol.

In some embodiments, the at least one protein or polypeptide or therapeutic compound is trypsin at a dosage of from about 10 mcg to 50 mcg per 99 g of cream or serum.

In some embodiments, the at least one protein or polypeptide or therapeutic compound is lysozyme, mexidole, a cannabinoid, reservatrol, insulin, chlorhexidine or combinations thereof.

In some embodiments, the combination comprises trypsin and lysozyme.

In some embodiments, the combination comprises trypsin and insulin.

In some embodiments, the combination comprises resveratrol and curcumin or reservatrol and lysozyme.

This invention provides, in some embodiments, a method for preparing a composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group;
Said method comprising the step of:
activating cotton fibers containing material comprising cellulose via application of a solution containing aldehyde functional groups to achieve oxidized cellulose components of said fibers thereof;
pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier;
applying a solution of one protein or polypeptide or therapeutic compound to said activated textile; and
drying said activated textile; and
Grinding or milling said activated textile to obtain oxidized cellulose microparticles carriers of between about 7-20 μm in diameter and of between about 0.5-100 μm in length.
Wherein a minority of about 20% of said protein or polypeptide or therapeutic compound is covalently bound and a majority of about 80% of said protein or polypeptide or therapeutic compound is non-covalently associated with said cellulose microparticles carriers.

This invention provides, in some embodiments, a method for improving stability of a protein, polypeptide or therapeutic compound in a composition for delivery to a mucosal surface of a subject, said composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group;
Said method comprising the step of:
activating cotton fibers containing material comprising cellulose via application of a solution containing aldehyde functional groups to achieve oxidized cellulose components of said fibers thereof;
pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier;
applying a solution of one protein or polypeptide or therapeutic compound to said activated textile; and
drying said activated textile; and
Grinding or milling said activated textile to obtain oxidized cellulose microparticles carriers of between about 7-20 μm in diameter and of between about 0.5-100 μm in length.
Wherein a minority of about 20% of said protein or polypeptide or therapeutic compound is covalently bound and a majority of about 80% of said protein or polypeptide or therapeutic compound is non-covalently associated with said cellulose microparticles carriers; and
Whereby said protein or polypeptide or therapeutic compound agent stability in said composition is improved as compared to existing compositions containing said agent.

This invention provides, in some embodiments, a method for improving a release profile of a protein, polypeptide or therapeutic compound in a composition for delivery to a mucosal surface of a subject, said composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group;
Said method comprising the step of:
activating cotton fibers containing material comprising cellulose via application of a solution containing aldehyde functional groups to achieve oxidized cellulose components of said fibers thereof;
pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier;
applying a solution of one protein or polypeptide or therapeutic compound to said activated textile; and
drying said activated textile; and
Grinding or milling said activated textile to obtain oxidized cellulose microparticles carriers of between about 7-20 μm in diameter and of between about 0.5-100 μm in length.
Wherein a minority of about 20% of said protein or polypeptide or therapeutic compound is covalently bound and a majority of about 80% of said protein or polypeptide or therapeutic compound is non-covalently associated with said cellulose microparticles carriers; and
   Whereby said protein or polypeptide or therapeutic compound agent is released in a sustained release model from said composition for at least a period of about a few hours following application.

In some embodiments, grinding or milling provides a powder, which may be incorporated in a tablet, capsule or sachet formulation. In some embodiments, grinding or milling provides a solid product that may be incorporated in a food product or dietary supplement. In some embodiments, grinding or milling provides a product for use in a topical composition and in some embodiments, the topical composition is a cream, mousse, serum, shampoo, conditioner or makeup. In some embodiments, the composition is for oral administration and in some embodiments, the composition is a tablet, capsule, powder, food product, syrup or liquid.

In some embodiments, the at least one protein or polypeptide or therapeutic compound comprises trypsin, insulin, curcumin or reservatrol and in some embodiments, the at least one protein or polypeptide or therapeutic compound comprises a cannabinoid. In some embodiments, the least one protein or polypeptide or therapeutic compound comprises a superoxide dismutase. In some embodiments, the at least one protein or polypeptide or therapeutic compound comprises glatiramer acetate (copaxone). In some embodiments, the at least one protein or polypeptide or therapeutic compound comprises trypsin, reservatrol, curcumin, insulin, lysozyme, or a combination thereof.

In some embodiments, the at least one protein or polypeptide or therapeutic compound is trypsin at a dosage of from about 10 mcg to 50 mcg per 99 g of cream or serum.

In some embodiments, the at least one protein or polypeptide or therapeutic compound is lysozyme, mexidole, a cannabinoid, reservatrol, insulin, chlorhexidine or combinations thereof.

In some embodiments, the combination comprises trypsin and lysozyme.

In some embodiments, the combination comprises trypsin and insulin.

In some embodiments, the combination comprises resveratrol and curcumin or reservatrol and lysozyme.

### BRIEF DESCRIPTION OF THE DRAWINGS

FIG. 1 plots a DISTRIBUTION CURVE of Microparticles Carriers comprising e.g., Trypsin of Bovine source made by a Vortex milling machine according to the present invention.

### DETAILED DESCRIPTION OF THE INVENTION

While the invention will now be described in collection with certain preferred embodiments in the following examples so that aspects thereof may be more fully understood and appreciated, it is not intended to limit the invention to these particular embodiments. On the contrary, it is intended to cover all alternatives, modification and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of proving what is believed to be the most useful and readily understood description of formulation procedures as well as of the principles and conceptual aspects of the invention.

The object of this invention is to eliminate the above-mentioned deficiency by creating tablets and capsules from based compound materials of dialdehyde cellulose (DAC) microparticle carriers and bioactive substances having releasable therapeutic activities, capable of passing intact the stomach, entering through the wall of the intestine into the bloodstream and doing the job effectively. and,

Creating creams containing bioactive substances such as proteins or chemicals and/or combinations thereof.

According to the present invention the based components in the form of dry powder of the bio-active tablets, capsules and the bio-creams are considerably cellulose (cotton gauze) microparticles varies between (Dv 1) 0.675-(Dv 100) 97.2 μm in which the median (Dv 50) is 10.2 μm, Dv 90 is 35.5 μm and Dv 99 is 69.3 μm.

Said carriers contains dialdehyde functional groups varies respectively from 0.208% w/w corresponding to 0.026 mg-equiv. up to 12% w/w corresponding to 1.5 mg-equiv. of dialdehyde groups per gram of carrier (DAC powder/DAC carriers). Some of the tablets, capsules and the creams comprising at least one selected bioactive enzyme or another bioactive substance, or further optionally comprising respectively more than one bioactive substance of different selected molecules from animals, flora or synthetic chemical groups origins, e.g. Trypsin, insulin, Lysozyme, Curcumin, Resveratrol, Cannabis, Mexidole, CHX, Ionic Silver, Vit. E and/or other bioactive substances or, combinations chemically immobilized and bound thereon by a combination of about 20% covalently bound and about 80% same or other bio-active substance bound by a dissorption process. Due to the nature of the bounding as per the present invention 95%-97% of the therapeutical agent is released fast and effective as native species—meaning the final products are provided with a reliable effective releasable curve.

One of the preferred aspects of the present invention is medical tablets and capsules and dietary supplements tablets and capsules, orally administered, which are aimed to combat diseases, disorder and disfunction. For example:

Trypsin tablets—trypsin breaks down proteins and it has the ability to break down the viruses' protein formation consequently neutralizing the viruses and reducing its risks. So, trypsin tablets are targeted to battle the coronavirus SARS CoV2 the cause of the “COVID-19” pandemic and other viruses of the corona family

Insulin tablets that is a means of lowering the blood sugar level; or,

Curcumin tablets/capsule—to strengthen the immunity system, Resveratrol tablets or capsules—antioxidant product or any product comprising any protein or any bio-active substance.

Another aspect of the present invention is tablets/capsules containing existing drugs orally administered replacing administration by injection, e.g. Glatirmer acetate (copaxsone).

Another preferred aspect of the present invention, that will be discussed hereinafter, is medical and cosmetic bioactive creams for treatment of human patients and animals.

Referring to some of the active ingredients:

### Insulin

Insulin an anabolic peptide hormone was found to be quite a large protein (monomer) of amino acid and has a molecular mass of 5808 kDa. The amino acid sequence of insulin is strongly conserved and varies only slightly between species. Bovine insulin differs from human in only three amino acid residues and porcine insulin in one. So, in other words it is similar enough to human to be clinically effective in humans. Biosynthetic insulin (rDNA, INN) for clinical use is manufactured by recombinant DNA technology

### Trypsin

Trypsin an enzyme cleaves peptide chains mainly at the carboxyl side of the amino acids, catalyzes the hydrolysis of peptide bonds and breaking down protein. Trypsin has a molecule mass of 23.3 kDa

According to scientific literature the structure of the SARS CoV2 (the corona virus of “Covid-19”) is shaped by 4 protein formations: Spikes, Membrane, Viral Envelope, Nucleocapsid, which stores and delivers the RNA genome.

Whereby upon being in contact with the SARS CoV2 virus or, with any other viruses of the corona family the trypsin breaks down its proteins formation subsequently neutralizing the virus and reducing its risk

### Curcumin

Curcumin is a bright yellow chemical extracted from Curcuma longa plants. It is the principal curcuminoid turmeric, a member of the ginger family Zingiberaceae. It is sold as a herbal supplement, cosmetic ingredient, food flavoring and food coloring.

### Lysozyme

Lysozyme is a naturally occurring enzyme found in bodily secretions such as tears, saliva, and milk. It functions as an antimicrobial agent by cleaving the peptidoglycan component of bacterial cell walls, which leads to cell death.

According to Helal R, et al., lysozyme has other properties aside immunity; it acts against viruses, inflammation and cancer.

### Resveratrol

Resveratrol (3,5,4?-trihydroxy-trans-stilbene) is a stilbenoid, a type of natural phenol, and a phytoalexin produced by several plants in response to injury or when the plant is under attack by pathogens, such as bacteria or fungi. Resveratrol in food include the skin of grapes, blueberries, raspberries, mulberries and peanuts. Commonly used as a dietary supplement as an antioxidant agent.

### Superoxide Dismutase

Superoxide dismutase (SOD) is an enzyme that alternately catalyzes the dismutation (or the partitioning) of superoxide (02?) radical into ordinary molecular oxygen (O2) and hydrogen peroxide (H₂O₂). Superoxide is produced as a by-product of oxygen metabolism and if not regulated, causes many types of cell damage. Hydrogen peroxide is also damaging and is degraded by other enzymes such as catalase. Thus, DOD is an important antioxidant defense in nearly all living cells exposed to oxygen.

Cannabis—CBD and/or THC

### Mexidole

Mexidole (another name: Emoxypine) is an antioxidant. According to the Russian Pharmacopeia it has a wide range of applications in medical practice. It properties exercise anxiolytic, anti-stress, anti-alcohol, anti-convulsant, nootropic, neuroprotective and anti-inflammatory action. Mexidole presumably improves cerebral blood circulation, inhibits thrombocyte aggregation, lowers cholesterol levels has cardioprotective and anti-atherosclerotic action.

Said cellulose microparticle carriers comprising bioactive substance, i.e., the active ingredients of the tablets, capsules and the bio-creams, having releasable therapeutically activities properties are being obtained by the following technological cycle:

Incorporating into cellulose cotton or viscose textile fabrics (carriers) dialdehyde groups respectively per the designed oxidation degree in order to get the suitable DAC carrier(s) as to ensure capturing the relevant amount of the desired specific bioactive substance(s).

Capturing bioactive substance, e.g. insulin or more bioactive substances e.g., insulin or trypsin or any other protein or synthetic chemical substance(s) into the volume of the initial material carriers.

Forming, from said cellulose cloth-carrier materials, by a continuous micronizing milling process microparticle carriers in the shape of a dry powder state which comprising bio-active substance(s) corresponding to the final required therapeutic activity.

Forming from said microparticle materials tablets or capsules comprising e.g., insulin or trypsin or any other bioactive substances. and,

Forming bio-creams by adding into cream(s) a designed amount of said microparticle material(s) which comprising specific bioactive substance(s), as described hereto, due to the required therapeutic activity, and mixing it properly until getting a smooth homogeneous texture.

More specifically, the fabric is immersed at room temperature in a solution, which contains considerably functional groups varies from 0.208% w/w corresponding to 0.026 mg-equiv. up to 12% w/w corresponding to 1.5 mg-equiv. of dialdehyde groups per gram of carrier. The proposed range of dialdehyde cellulose (DAC) carriers is suitably designed so as to meet the requirements of the active components of the present bioactive solutions and especially to enable the binding of said bioactive components of the herein required ratio and to ensure the appropriate release of said components as described hereinafter respectively to the desired therapeutical activity of the final designed product. In a continuous process the excess solution is pressed out and the carrier material(s) is dried utilizing a wringer with two or three rollers at 25 Kg-35 Kg pressure per 1 sq. cm. The excess solution is pressed out spreading the active compounds uniformly into the volume of the textile carrier and on the surface of the fabric. Thus, the fabric is partially dried. The carrier material(s) is further dried by utilizing a dryer, i.e., a hot air blowing chamber, a hot dram chamber heated from about 35? C up to 100? C; a vacuum chamber or by being lyophilized. Then the material is immersed for 2 hours in a tub containing a solution of trypsin, or insulin or any other biological or flora or chemical substances at a ratio of solution weight to carrier that varied respectively between 3.3 to 4.2 as described herein below in order to achieve maturation of the immobilization of the bioactive substance(s) impregnated thereon. The so-called maturation process of the treated said fabric during in which the completion of chemical immobilization is achieved is 2 hours only. During the immobilization process, about 20% of the bioactive substances are bound chemically to the carrier, and about 80% by sorption. Subsequently 95%-97% of the therapeutical molecules are releasable and effective as native species.

The following factors determine the dosage and efficacy of the tablets, capsules and the bio-creams:

The oxidation degree which limits the maximum quantity of bio-active substances that could be bound to the carrier(s), the variety and the number of bio-active substances which are bound to the carriers, and the amount of each of the bio-active substances per 1 gram of carrier, the amount/weight of microparticles per one tablet or capsule or 1 gram of bio-cream.

More specifically, as to get according to the present invention a trypsin tablet, e.g., of 200 mg that will contain 2 mg trypsin or a trypsin tablets of 250 mg that will contain 2.5 mg trypsin, the solution for activating by immersion the cloth-carrier(s) with trypsin, respectively to 1.0 kg of cellulose textile cloth-carrier(s) having oxidation degree of 1.5%. should contain 10 grams of trypsin. The cellulose carriers all along the way including the microparticles, i.e., the components that compose the tablets and capsules or will be used as the active ingredient of the bio cream will contain 1.0% of trypsin. Consequently, tablets of 200 mg made of said materials will contain 2 mg of trypsin and tablets of 250 mg will contain 2.5 mg of trypsin. Any cream or serum containing 1 g of said bio-active materials will contain 10 mg of trypsin per 100 g of cream.

In preferred embodiments of the present invention, e.g., as to get a trypsin tablet of 200 mg that will contain 4 mg trypsin or trypsin tablets of 250 mg that will contain 5 mg trypsin the solution for preparing final products: tablets, capsules should contain 20 grams of trypsin respectively per 1 (one) kg cellulose textile carrier having oxidation degree of 6.0%. Consequently, the resulted cellulose carriers, all along the way, will comprise 2.0% trypsin means all the components for putting together the final products—trypsin tablets of 200 mg made of said materials will contain 4 mg trypsin and trypsin tablets of 250 mg will contain 5 mg trypsin.

In other preferred embodiments of the present invention as to get trypsin tablets of 200 mg each that will contain 10 mg trypsin or trypsin tablets of 250 mg that will contain 12.5 mg trypsin, The trypsin solution for preparing the final products: tablets, capsules shall contain 50 grams trypsin respectively per 1 Kg of cellulose textile cloth-carrier(s) of 6.0% oxidation degree.

The resulted carriers all along the way including the microparticle for putting together the final products is comprising 5.0% trypsin, which results in higher therapeutic activity. Consequently, a trypsin tablet of 200 mg made of said materials will contain 10 mg trypsin and a trypsin tablets of 250 mg will contain 12.5 mg trypsin.

In special preferred embodiments of the present invention as to get a trypsin tablet of 200 mg that will contain 20 mg trypsin and a trypsin tablets of 250 mg that will contain 25 mg trypsin the trypsin solution for preparing the active components of the tablets, capsules and the bio-creams should contain 100 g of trypsin respectively per 1 (one) kg textile carrier having 12.0% oxidation degree. Thus. the resulted carriers all along the way including the microparticle carries, the components for putting together the final products, are comprising 10.0% trypsin, i.e., 100 mg per 1 g of carrier. Consequently, trypsin tablets of 200 mg made of said materials will contain 20 mg trypsin and a trypsin tablets of 250 mg will contain 25 mg trypsin.

It should be indicated that trypsin in any form should be stored at very cold temperatures between −20 to −80? C to prevent autolysis.

However, the trypsin microparticles carriers which were made according the present invention were stored at room temperature 15-25° C. and was activated after 10 years. Its efficacy was comparable to native samples. Thus, its shelf life at room temperature could be considered at least 10 years. Trypsin tablets and capsules made according to the present invention entirely preserves same properties aforesaid and its shelf life could be considered same. Bio-creams in which said microparticles are its active ingredient(s) self-life is at least 3 years.

### Insulin

In another preferred embodiments of the present invention the bio-active solution for immersion 1 kg of textile cloth material of 6.0% oxidation degree aimed for preparing microparticles carriers comprising, e.g., insulin 3.637 g insulin per 1 Kg Textile cloth. The obtained carriers contain 3.5 mg of insulin equivalent to 100 units of insulin (U-100) per 1 g of carrier. Consequently, 5 tablets made of 1 gram of said microparticles carriers, each tablet weighing 200 mg and containing U-20 per tablet.

In another preferred embodiment of the present invention the bio-active solution for immersion 1 kg of textile carrier of 6.0% oxidation degree for preparing the insulin tablet contain 7.274 g insulin. The obtained carriers contain 7 mg of insulin equivalent to 200 units (U) of insulin per 1 g of carrier Consequently, 5 tablets prepared from said microparticles carriers each tablet weighing 200 mg and containing 40 U of insulin per tablet.

After the immersion of the textile carrier during 2 hours in the bio-active solution and accomplishing maturization of the bio-active substance(s) (e.g. trypsin or, insulin or any other substance(s) bound thereof, the impregnated fabric is then pressed out by a wringer between two rollers. The ingredients hereinabove described are equally and uniformly incorporated and spread into the volume of the textile carrier and onto the surface of the fabric, while the excessive solvents are pressed out. The obtained material is again dried by utilizing forced hot air, oven drying at 20° C. up to 35° C., or regarding bioactive substances which are not enzymes or proteins up to 100° C. respectively, or vacuumed in a vacuum chamber and/or lyophilized at −15° C. to −10° C. The speed of the drying process is synchronized.

Transformation of the cellulose cloth-carrier material to microparticles carriers in the form of a dry powder was done by a grinding—milling machine, which creates a vortex flow, i.e., a type of a tornado storm. In using such a machine, aerodynamic forces are the main driver for the micronization process in getting the requested carriers' sizes. The Vortex mill(s), which require an air supply at a pressure of up to 6 bars, creates those aerodynamic conditions similar to the conditions in a tornado storm. The particles (i.e., the cotton fibers) breaks along its weak points when it is exposed to certain aerodynamic conditions.

The Vortex mill comprises a compact vortex chamber with no moving parts, which is unlike other milling technologies that are based on mechanical forces.

The principle is also different than in other air-jet mills: this technology is based on subjecting the particle to rapid changes in air pressure and flow directions. Hence, the particle is breaking along its natural dents, cavities or crystal defects. The result is a “clean cut” that is not accompanied with compression or amorphization of crystal surface typical to standard jet-mills. This is a “soft milling” process providing:
No Trauma to the material
Protection from degradation or amorphization
Handling soft, elastic substances such as textile carriers' materials.

Subsequently the natural properties of the textile carriers' materials and the added—acquired properties are being preserved.

The special airflow dynamics in the system imply that the lowest air pressure point is located within the micronizer. There is no Venturi injector or inlet hoppers that could clog.

The milling process of the Vortex mill is a continuous process. The final milling results are achieved in one milling stage. There is no need for multi-pass milling.

By using the Vortex milling machine said basic bio-active textile cloth material was micronized to microparticles bioactive carriers on the form of dry powder on the order of 0.675-97.2 μm having a median*(Dv 50) of 10.2 μm.
The median (Dv 50) of 10.2 μm is according to the results as per the Gaussian Curve.

It should be indicated that less milling of the textile cloth material(s) will yield bigger microparticles. The more milling will be resulting smaller microparticles. The variance of the distribution of said microparticle carriers follows the Gaussian normal distribution curve as described hereto under.

DISTRIBUTION CURVE of Microparticles Carriers comprising e.g., Trypsin of Bovine source made by a Vortex milling machine according to the present invention

The analysis was conducted using a Malvern Laser 3000 Instrument.

So, the present invention inter alia provides a method for preparing cellulosic microparticle carriers on the order between 0.675-97.2 μm in which the median (Dv 50) is 10.2 μm and creating from said microparticles carriers' pharmaceutical tablets for humans and animals' treatment weighing 100 mg, 200 mg or, 250 mg (or even more) containing different doses of a bioactive substance or a plurality of bio-active substances.

Tablets were made by using RONCHI machines or a KORSCH machine having a max compressing force of 100 KN.

Tablets of 2 mm depth fill and 8 mm diameter, was made by using a Carver Pressing Machine having a compression force of 580 Kg.

Tablets of 2/17 mm depth fill and up to 11 mm diameter, was made by using a EA RONCHI machine by using compression range of maximum 5,000 kg.

Tablets of 2/17 depth fill and 6-18 mm diameter were made by using a KORSCH having a max compressing force of 100 KN by using compression range of maximum 5,000 kg.

Pharmaceutical tablets for humans' and animals' treatment as well of 2/17 mm depth fill and up to 25 mm diameter was made by using a PA RONCHI machine having a max compressing force of 100 KN by using compression range of maximum 5,000 kg. It could be done by any other suitable/similar compression machines.

In the middle of each tablet there is a slot that enables splitting the tablet to two equal parts easily so it could be administered respectively twice.

It should be indicated that the suggested range of dialdehyde groups within the carriers according to the present invention enables binding various amounts of bio-active substance(s) thereon. Consequently, creating a variety of doses per 1 gram of carrier.

The final dosage and the dimension of a tablet derive of the ratio between the following factors: The oxidation degree and the corresponding dosage level of the bio-active substance(s) comprised within the microparticles carriers, which is required as to achieve the desired effectiveness of the specific tablet(s).

Another preferred embodiment of the present invention is medicinal tablets like candies of 2/17 mm depth of fill and diameter sizes up to 25 mm. The medicinal tablets like candies containing said cellulose microparticles carriers comprising at least one bioactive substance or a variety of different molecules of bioactive substances. The medicinal candy pills are being made by a candy powder press machine with a rotary tablet press of maximum pressure 40 KN or a PA RONCHI tablet press machine.

It should be noted that 1 g of microparticles can be produce 10 tablets each weighing 100 mg and respectively 5 tablets each weighing 200 mg, 4 tablets each weighing 250 mg, or 2 tablets each weighing 500 mg and also to produce 1 tablet weighing 800 mg, etc.

### Pharmaceutical and Dietary Supplement Capsules

According to the scientific literature, Covid-19 viruses are being spread by aerosol and infecting humans by entering the body mainly via the respiratory system—nose and throat—and then spread itself into the lungs and other organs of the body. Trypsin medicinal candies containing trypsin 2%-5% w/w and/or also various dosages are aimed to battle the viruses. During the sucking process of the trypsin candy the trypsin molecules are being released within the throat. While being in direct contact with the coronavirus spread in the throat the trypsin is breaking down the viruses' proteins and reducing its risk.

Another preferred aspect of the present invention is pharmaceutical and dietary supplement capsules containing said microparticles carriers comprising at least one bioactive substance or a variety of different molecules of bioactive substances.

The capsules are made by using a capsule powder filling machine (suction 1.0 KW and vacuum—1.5 KW) having accurate operation for orientation, opening, filling, joining and ejecting of the capsules. A spiral feeding in the powder magazine hoop to ensure powder feeding and filling (compulsory). Dual capsule-opening design to ensure all capsules are opened. Electrical impact protection device to prevent further damage. The machine will stop automatically in the event of collision. Safety sensing system to shut the machine down when automatically in the event of problems when no capsules, no powder, door is opened and abnormal load for machine is detected. Automatic vacuum removal of the air in the dosing tube to give more accurate weight of the filled capsule (compulsory). Exclusive dosing tube system, temperature of the powder will not rise during the filling process (compulsory). Adjustable compression piston in dosing tube to adjust the powder volume without change parts. Or a similar capsule making machine.

Production is done as follows:

Hard gel capsules are applied in the requested size: 0, 1, 2, 3, 4 due to the desired final product. After capsules are arranged and oriented the capsules are placed into the mold and opened by vacuum, and then opened once again to ensure the capsule to be open. The machine ejects the unopened capsules and the faulty into a collection box. The dosing tube put into powder place. The powder is compressed into slug. Then the powder slug is ejected into the open capsule. The machine reclaims the powder which not being filled into the capsule from the collector. Capsules parts (caps and body) are being joined together by upper and lower closing pins. the filled capsules are ejected into the collection container. The residue powder attached to cap and body mold is cleaned by vacuum suction for next cycle.

### Trypsin Tablets

One embodiment of the tablets of 200 mg contains cotton microparticles carriers on the order of 0.675-97.2 μm, having 3% dialdehyde groups corresponding to 0.375 mg-equiv per 1 g of the textile carrier comprising 4 mg trypsin per 1 tablet

One of the embodiments of tablets of 200 mg made according to the present invention contains cotton microparticles carriers on the order of 0.675-97.2 μm, having 6% of dialdehyde groups corresponding to 0.75 mg-equiv., comprising 8 mg trypsin per 1 tablet

One of the embodiments of the tablets of 200 mg made according to the present invention contains cotton microparticles carriers on the order of 0.675-97.2 μm having 6% of dialdehyde groups corresponding to 0.75 mg-equiv. comprising 10 mg trypsin per 1 tablet.

Another embodiment of the tablets of 250 mg made according to the present invention contains cotton microparticles carriers on the order of 0.675-97.2 μm having 9% of dialdehyde groups corresponding to 1.125 mg-equiv. dialdehyde groups, comprising 16 mg trypsin per 1 tablet.

One preferred embodiment of the tablets of 250 mg made according to the present invention contains cotton microparticles carriers on the order of 0.675-97.2 μm, having 12% of dialdehyde groups corresponding to 1.5 mg-equiv. dialdehyde groups, comprising 20 mg trypsin per 1 tablet.

In another aspect of the present invention—capsules, one embodiment of capsules contains cotton microparticles carriers on the order of 0.675-97.2 μm weighing 200 mg, having 0.125 mg-equiv. (1.0%) dialdehyde groups comprising 4 mg (2% w/w) trypsin per 1 capsule.

One embodiment of the present invention is a capsule contain cotton microparticles carriers on the order of 0.675-97.2 μm, weighing 200 mg having 0.1875 mg-equiv. (1.5%) dialdehyde groups comprising respectively 2 mg (1% w/w) trypsin and 4 mg (2% w/w) curcumin per 1 capsule.

Another embodiment of the present invention is a capsule contain cotton microparticles carriers on the order of 0.675-97.2μ, having 0.75 mg-equiv. (6%) dialdehyde groups, weighing 300 mg comprising respectively 5% w/w (15 mg) trypsin and 10% w/w (30 mg) curcumin per 1 capsule.

A preferred embodiment of capsule of the present invention contains cotton microparticles carriers on the order of 0.675-97.2 μm, weighing 300 mg having 0.75 mg-equiv. (6%) dialdehyde groups comprising respectively 5% w/w (15 mg) cannabis and 3% w/w (9 mg) resveratrol per 1 capsule.

Another embodiment contains 2% trypsin plus 5% resveratrol w/w.

Some preferred embodiments contain 5% w/w trypsin and 0.5% w/w lysozyme, or trypsin 5% plus curcumin 10% w/w.

Another preferred embodiment in the form of powder named blue, weighing 5 g per unit, contains (w/w): trypsin 5%, curcumin 25%, ginger 25%, arabic coffee respectively up to 100%

Another preferred embodiment weighing 2.5 g, contains: Trypsin microparticles carriers—50 mg; isomaltose—1575 mg; xylitol—355 mg; carbon dioxide—175 mg; natural peppermint—140 mg; caffein—100 mg; coconut oil 92.5 mg; natural flavor—7.5 mg; zinc—2 mg.

Another preferred embodiment weighing 2.5 g, contains: resveratrol microparticles carriers—50 mg; trypsin microparticles 25 mg; curcumin—35 mg; sugar 1,100 mg; lactose—365 mg; corn syrup solids—365 mg; carbon dioxide—247.5 mg; natural and artificial flavors—82.5 mg; caffeine—80 mg; coconut oil—100 mg; malic acid—45 mg; zinc—2.5 mg. sucralose—2.5 mg.

It should be indicated that a variety of capsules containing various bio-substances at different dosage could be made according to the present invention. The dosage of the capsules derives of the ratio between the dosage level of the bio-active microparticles carriers {i.e., the components of the capsule(s] and the amount of the components which required as to achieve the desired effectiveness of the capsule(s).

### Insulin Tablets

One preferred embodiment of the present invention is insulin tablets orally administered, aimed to reduce glucose level in the blood, weighing 100 mg, or 200 mg, or 250 mg or 500 mg or more containing different doses of insulin units varied from 10 unit to 200 units per tablet, derived of the insulin dosage per 1 g of the microparticles carriers.

One of the embodiments of the tablets made according to the present invention contains cotton microparticles carriers on the order of 0.675-97.2 μm, having 0.05 mg-equiv. dialdehyde groups, weighing 200 mg each comprising 10 U insulin units per tablet.

One of the embodiments of the tablet(s) made according to the present invention contains cotton microparticles carriers on the order of 0.675-97.2 μm, having 0.125 mg-equiv. dialdehyde groups, comprising 20.0 U of insulin per tablet.

One preferred embodiment of the insulin tablets made according to the present invention contains cotton microparticles carriers on the order of 0.675-97.2 μm, having 0.375 mg-equiv. dialdehyde groups, comprising insulin 50.0 U insulin per tablet.

Another preferred embodiment of the tablet(s) made according to the present invention contains cotton microparticles carriers on the order of 0.675-97.2 μm, having 0.75 mg-equiv. dialdehyde groups, comprising 60.0 U insulin per tablet.

Another preferred embodiment of the tablet(s) made according to the present invention contains cotton microparticles carriers on the order of 0.675-97.2 μm, having 0.75 mg-equiv. dialdehyde groups, comprising 80.0 U insulin per tablet.

Additional preferred embodiment of the tablet(s) made according to the present invention contains cotton microparticles carriers on the order of 0.675-97.2 μm, having 0.75 mg-equiv. dialdehyde groups, comprising 100.0 U insulin per tablet.

One embodiment of the present invention is a tablet contain cotton microparticles carriers on the order of 0.675-97.2 μm, weighing 100 mg having 0.1875 mg-equiv. (1.5%) dialdehyde groups comprising on same carrier respectively 10 mg trypsin and 20 U insulin per 1 tablet.

Another preferred embodiment of the present invention is a tablet contain cotton microparticles carriers on the order of 0.675-97.2 μm, weighing 200 mg having 0.75 mg-equiv. (6%) dialdehyde groups comprising on same carrier respectively 10 mg (5% w/w) trypsin and 60 U insulin per 1 tablet.

One embodiment of the present invention is a tablet contain cotton microparticles carriers on the order of 0.675-97.2 μm, weighing 200 mg having 0.75 mg-equiv. (6%) dialdehyde groups comprising on same carrier respectively 20 mg trypsin (10%) and 100 U insulin per 1 tablet.

This invention provides the following contemplated embodiments, in addition to the summary and claims provided herewith:

### Bio-Creams and Serums

Another preferred aspect of the present invention is preparing bio-creams and serums for medical and cosmetic uses for treating wounds and aging disease of human patients, e.g., healing acne, managing and reliving psoriasis and seborrhea, antioxidant treatment, facial and body peeling; and for treatment of animals' wounds.

It should be indicated that the quantity of the bio-active substances within the creams or the serums derived of the ratio between the following factors: the dosage level of the bio-active microparticles carriers, i.e., the bio-active components of the cream(s)/serum(s), and the amount of the components added to the cream(s)/serum(s) as required to achieve the desired effectiveness.

Anti-Aging solutions on the market can be divided to 3 categories.
Chemical-based peeling products—creams and serums.
Natural component-based products
Laser treatment

Most of these products do not work as claimed.

Use of high concentration is a prerequisite in effective chemical peeling but, with that comes the risk of damage to the skin mainly in facial or other sensitive tissue. In order to reduce the risk companies recommending that 7-9 minutes after applying the cream on the face and rubbing the face the user must wash the face carefully and apply a facial cream. A consequence of limiting the time of use requires that it be repeated again and again.

In contrast, chemicals at low concentration are risk-free but ineffective. The market solution for face peeling, in order to minimize risk is out-of-home cosmetic treatment, at the hand of cosmeticians—a lucrative but expensive time-consuming venture.

The most eminent example for the Natural Component-based Products segment might be products made by Jeunesse Global (JB). JB products are based on stem cells, natural ingredients and vitamins, applied for DNA repair, dissolving of necrotic cells and building new tissue. JB claim the timespan for a treatment to be 6-9 hours.

Laser treatments can cause skin burns. Are costly demanding expert treatment. Are time and effort consuming as they are performed out of house.

The creams and serums as per the present invention are easy and immediate self-appliance anywhere anytime.

After applying the cream or the serum the user is free of washing face or body. The active ingredient in the cream can perform provided it is in a minimal humidity environment. The moisturizing environment is provided by the carriers—the creams or serums. As long as the carrier provides moisture the trypsin or any other active ingredient is active.

In some preferred embodiments of cream, the trypsin and/or other active ingredients are active even during 24 hours. The bioactive activity, e.g., the peeling process which is being done by the trypsin is performed as long as the moisture on the skin is present.

Serums containing bio-active ingredients, e.g., trypsin have a short duration of activity 10-30 min.' which derived of the serums' “carriers” properties. Surprisingly it turned out to us that the short-time activity of peeling is fast, strong and effective in removing necrotic tissues including scabs even at low concentration of the trypsin.

In addition, the trypsin-serum(s) dries the wound site and surprisingly the scarring is lower if at all.

No side effects with trypsin cleaving and dissolving necrotic tissues only. Synergy effect in using creams or serums comprising more than one bio-active agent.

The bio-creams and bio-serums are made by adding, e.g., bioactive cellulose microparticles carriers of 0.675-97.2 μm, containing 0.1875 mg-equiv. dialdehyde groups and comprising 1% w/w trypsin to any cream or serun, at the ratio of 1.0 g of said microparticles to 99 g of cream or serum (total weight: 100 g of cream/serum, mixing it slowly with a mixer about 2-5 min until getting a smooth homogeneous texture. The dosage of the trypsin within the obtained products is 10 mg per 100 g cream.

In preferred embodiment of the creams or the serums made according to the present invention, 1 g. of said microparticles carriers having oxidation degree of 1.5% respectively 0.1875 mg-equiv. containing 3% w/w trypsin is added to 99 g (total 100 g) of any cream or any serum. The dosage of the trypsin within the obtained products is 30 mg (0.03%) per 100 g cream or serum.

In other preferred embodiments of the creams and/or the serums made according to the present invention 1 g of said microparticle carriers containing oxidation degree of 0.75 mg-equiv. and trypsin dosage of said microparticles is of 5% or 7% w/w is added to 99 g of cream or serum made according to the present invention. The dosage of the trypsin within the creams/serum is 50 mg (0.05%) or 70 mg (0.07%) respectively per 100 g of cream or serum.

In special preferred embodiment of the creams and/or the serums made according to the present invention 1 g of said microparticles carriers oxidation degree of said microparticles carriers is 12% (1.5 mg-equiv.) and trypsin 10% w/w is added to 99 g of cream or serum. The resulted cream/serum contains 100 mg (0.1%) trypsin.

In another preferred embodiment of the creams and/or the serums made according to the present invention, 100 g of cream or serum contains 1 g microparticles carriers of 0.675-97.2 μm, having 0.125 mg-equiv. of dialdehyde groups comprising 10 mg to 50 mg cannabis.

In other preferred embodiments of the creams and/or serums made according to the present invention 100 g of cream or serum contains 1 g microparticles carriers of 0.675-97.2 μm, having 0.125 mg-equiv. of dialdehyde groups comprising SOD between 10 mg to 50 mg.

In another preferred embodiment of creams or serums made according to the present invention 100 g of cream contains 1 g microparticles carriers of 0.675-97.2 μm, having 0.1875 mg-equiv. of dialdehyde groups comprising 2% w/w chlorhexidine, i.e., 20 mg chlorhexidine (CHX).

Another preferred embodiment of creams or serums made according to the present invention contains two bio-active substance or a plurality of bioactive substances: 100 g of cream or serum contain 1 g of microparticles carriers of 0.675-97.2 μm, having 0.75 mg-equiv. dialdehyde groups comprising 0.01 g trypsin and 0.0033 g resveratrol.

Another embodiment of the creams and/or the serums made according to the present invention 100 g contains 1 g of microparticles carriers of 0.675-97.2 μm, having 0.75 mg-equiv. dialdehyde groups comprising 0.01 g trypsin and 0.01 resveratrol.

Another embodiment of the creams and/or the serums made according to the present invention 100 g contain microparticles carriers of 0.675-98.1 μm, having 0.75 mg-equiv. dialdehyde groups comprising 0.01 g cannabis and 0.0033 g (3.3 mg) mexidole.

Another embodiment of the creams and/or the serums made according to the present invention 100 g contains 1 g of microparticles carriers of 0.675-97.2 μm, having 0.75 mg-equiv. dialdehyde groups comprising 0.01 g curcumin and 0.01 g resveratrol

Another embodiment of the creams and/or the serums made according to the present invention 100 g of cream contains 1 g of microparticales carriers of 0.675-97.2 μm μm, having 0.75 mg-equiv. dialdehyde groups comprising 0.01 g. trypsin, 0.01 g insulin and CHX 2% w/w (20 mg) per 1 gram of said microparticles carriers.

From the aforesaid method for the calculation of the hydrolysis of protein and/or other bioactive substances, incorporated in a dialdehyde cellulose carrier as it is released from the carrier itself, it will be understood, as per the equation of rate of release, that because of the sorption immobilization 95%-97%, almost all of the enzyme(s) and/or any another bioactive protein substance(s) or any synthetic chemical incorporated within the tablets, capsules or the active component of the creams is released effectively.

Still additional contemplated aspects of the invention include, but are not limited to:

A method for preparing various tablets containing oxidized cellulose microparticles carriers made of textile cellulose cotton gauze on the order between 0.675-97.2 μm, activated by immersing a textile carrier material in a solution containing functional groups to affect the oxidation thereof and pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier, impregnating said active carrier in a solution of at least one bioactive substance or other bioactive substances having therapeutic activity and drying the same. Then by a grinding/milling process forming (from said treated textile cloth) cellulose microparticles carriers on the order between 0.675-97.2 μm, in the form of powder in which a minority about 20% of said bioactive substance(s) is bound by covalent bonds and a majority about 80% is bound by sorption and then said powder is being transformed to pharmaceutical tablets, capsules or added to creams having therapeutical properties releasable in effective amounts.

Some aspects include where the activated carrier is impregnated in a solution of at least one bioactive enzyme and/or any bio-active substance of animal, flora or synthetic chemical origin. Some aspects include where the at least one bioactive substance is trypsin. Some aspects include where the at least one bioactive substance is insulin. Some aspects include where the at least one bioactive substance is curcumin. Some aspects include where the at least one bioactive substance is resveratrol. Some aspects include where the at least one bioactive substance is cannabis. Some aspects include where the at least one bioactive substance is SOD or any other bio-active substance of animal, flora or synthetic chemical origins. Some aspects include where the at least one bioactive substance is an existing drug—glatiramer acetate (copaxsone). Some aspects include where the more than one bioactive substance is an existing drug—glatiramer acetate (copaxsone) and chitosan. Some aspects include where the more than one bioactive substance is trypsin and insulin. Some aspects include where the more than one bioactive substance is resveratrol and curcumin.

This invention provides a method for preparing various capsules containing oxidized microparticles carriers made of textile cellulose cotton gauze carriers, on the order between 0.675-97.2 μm, activated by immersing a textile carrier material in a solution containing functional groups to affect the oxidation thereof and pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier, impregnating said active carrier in a solution of at least one bioactive substance or other bioactive substances having therapeutic activity and drying the same. Then by a grinding/milling process forming cellulosic microparticles carriers in the form of dry powder in which a minority about 20% of said bioactive substance(s) is bound by covalent bonds and a majority about 80% is bound by sorption and then said powder is being filled respectively into capsules or, being mixed with other components and the capsules contains bio-active cellulose microparticles carriers in the form of dry powder.

Some aspects include where the activated carrier is impregnated in a solution of at least one bioactive enzyme and/or any bio-active substance of animal, flora or synthetic chemical origin. Some aspects include where the at least one bioactive substance is curcumin. Some aspects include where the at least one bioactive substance is resveratrol. Some aspects include where the at least one bioactive substance is mexidole. Some aspects include where the at least one bioactive substance is superoxide dismutase (SOD). Some aspects include where the at least one bioactive substance is cannabis. Some aspects include where the activated carrier is impregnated in a solution of at least two bioactive substances. Some aspects include where the one bioactive comprised within the capsules is curcumin and the other one is resveratrol.

In some embodiments, this invention provides a method for preparing various dietary supplements containing oxidized microparticles carriers made of textile cellulose cotton gauze carriers, on the order between 0.675-97.2 μm, activated by immersing a textile carrier material in a solution containing functional groups to affect the oxidation thereof and pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier, impregnating said active carrier in a solution of at least one bioactive substance or other bioactive substances having therapeutic activity and drying the same. Then by a grinding/milling process forming cellulosic microparticles carriers in the form of dry powder in which a minority about 20% of said bioactive substance(s) is bound by covalent bonds and a majority about 80% is bound by sorption and then said powder is being filled respectively into capsules or, being mixed with other components and the capsules contains bio-active cellulose microparticles carriers in the form of dry powder.

Some aspects include where the microparticles contains is a combination in the form of powder of at least one bioactive substance and additional components. Some aspects include where the bioactive substance is resveratrol and curcumin, ginger and arabic coffee. Some aspects include where the bioactive substance is mexidole and curcumin, ginger and arabic coffee. Some aspects include where the textile bioactive substance is resveratrol and sugar, lactose, corn syrup solid, carbon dioxide, caffein, coconut oil, natural flavor, malic acid, sucralose and zinc. Some aspects include where the bioactive substance is mexidole and sugar, lactose, corn syrup solid, carbon dioxide, caffein, coconut oil, natural flavor, malic acid, sucralose and zinc. Some aspects include where the bioactive substance is resveratrol, trypsin, curcumin and sugar, lactose, corn syrup solid, carbon dioxide, caffein, coconut oil, natural flavor, malic acid, sucralose and zinc. Some aspects include where the bioactive substance is resveratrol and isomaltose, xylitol, carbon dioxide, peppermint flavor, caffein, coconut oil, natural flavor, and zinc. Some aspects include where the bioactive substance is mexidole and isomaltose, xylitol, carbon dioxide, peppermint flavor, caffein, coconut oil, natural flavor, and zinc. Some aspects include where the bioactive substance is resveratrol, trypsin, curcumin and isomaltose, xylitol, carbon dioxide, peppermint flavor, caffein, coconut oil, natural flavor, and zinc.

This invention also provides a method for preparing various bio-creams, bio-serums, bio-shampoo containing oxidized cellulose microparticles carriers of cotton gauze or textile knitted caproamide fibers, activated by immersing a textile carrier material in a solution containing functional groups to affect the oxidation thereof and pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier, impregnating said active carrier in a solution of at least one bioactive substance or other bioactive substances having therapeutic activity and drying the same. Then by a grinding/milling process said treated textile carrier forming cellulosic microparticles carriers on the order between 0.675-97.2 μm in the form of dry powder, in which a minority of said bioactive substance(s) about 20% is bound by covalent bonds and a majority about 80% by sorption is added respectively to any cream and then mixed until the mixture of the cream is homogeneous and smooth. The resulted cream which containing said bio-active microparticles carriers has therapeutical properties releasable in effective amounts.

Some aspects include where the activated carrier is impregnated in a solution of at least one bioactive enzyme and/or any bio-active substance of animal, flora or synthetic chemical origin. Some aspects include where the at least one bioactive substance is trypsin. Some aspects include where the at least one bioactive substance is lysozyme. Some aspects include where the at least one bioactive substance is resveratrol. Some aspects include where the at least one bioactive substance is mexidole. Some aspects include where the at least one bioactive substance is insulin. Some aspects include where the at least one bioactive substance is cannabis. Some aspects include where the at least one bioactive substance is superoxide dismutase. Some aspects include where the at least one bioactive substance is cannabis. Some aspects include where the at least one bioactive substance is chlorhexidine. Some aspects include where the micro-particles carriers contains two bioactive substances. Some aspects include where the bioactive substance are trypsin and lysozyme. Some aspects include where the more than one bioactive substance are trypsin and insulin. Some aspects include where the micro-particles carriers are three bioactive substances. Some aspects include where the d bioactive substance are trypsin, resveratrol and curcumin. Some aspects include where the bioactive substance are trypsin, resveratrol and lysozyme.

Some aspects of the invention include various solid pharmaceutical tablets orally administered containing oxidized cellulose microparticles carriers made of textile cellulose cotton gauze on the order between 0.675-97.2 μm, containing functional groups in the volume and on the surface of the carriers thereof, between about 0.208% w/w considerably 0.026 mg equiv. to 12.0% w/w considerably 1.5 mg-equiv. of aldehyde groups per gram of carrier, in various weights, which ranging between 100 mg up to 500 mg, comprising at least one bioactive substance or a composition of several bioactive substances of animal, flora origin or combination of bioactive substances and synthetic chemicals, having therapeutical releasable properties in various dosages considerable to tablets weights. In the middle of each tablet there is a slot that enables splitting the tablet to two equal parts easily, so it could be administered respectively twice.

Some aspects include where the one bioactive substance is trypsin in various weights. Wherein the trypsin's dosages vary considerably from 100 mg to 100 mg per 1 g of tablets. Some aspects include where the one bioactive substance is insulin in various weights, wherein the insulin dosages vary considerably from 100 U of insulin per 1 g of tablets up to 1,000 U of insulin per 1 g of tablets. Some aspects include where the one bioactive substance is curcumin in various weights wherein the curcumin's dosages vary considerably from 50 mg up to 125 mg per 1 g of tablets. Some aspects include where the one bioactive substance is resveratrol in various weights, wherein the resveratrol dosages vary considerably from 50 mg to 125 mg per 1 g of tablets. Some aspects include where the one bioactive substance is cannabis in various weights and the dosages vary from 50 mg to 125 mg per 1 g of tablets. Some aspects include where the one bioactive substance is SOD in various weights and dosages vary from 50 mg to 125 mg per 1 g of tablets. Some aspects include where the one bioactive ingredient is an existing drug glatiramer acetate (copaxsone). Dosages vary from 20 mg to 40 mg per tablet. Some aspects include where the one bioactive ingredient is any protein in various weights and dosages. Some aspects include where the two bioactive substances are trypsin and insulin in various weights and dosages, wherein the insulin dosages vary considerably from 100 U of insulin per 1 g of tablets up to 1,000 U of insulin per 1 g of tablet and the trypsin dosage from 20 mg to 100 mg per 1 gram of tablet. Some aspects include where the two bioactive substances are trypsin and lysozyme in various dosages, wherein the trypsin dosage vary considerably from 20 mg to 100 mg per 1 g of tablet and lysozyme from 5 mg to 10 mg per 1 g of tablets. Some aspects include where the two substances are Trypsin and resveratrol wherein the trypsin dosage vary considerably from 20 mg to 100 mg per 1 g of tablet and the resveratrol from 50 mg to 125 mg per 1 g of tablets. Some aspects include where the two substances are resveratrol and curcumin, wherein the dosage of the resveratrol vary considerably from 50 mg to 125 mg per 1 g of tablets and the curcumin from 200 mg to 400 mg per 1 g of tablets. Some aspects include where the bioactive ingredients are existing drug glatiramer acetate (copaxsone), wherein the dosages vary from 20 mg to 40 mg per tablet and 2 mg up to 5 mg chitosan per 1 tablet. Some aspects include where the two or more bioactive ingredient are any protein or combination of proteins or compositions of proteins and synthetic chemicals ingredients in various dosages.

This invention also contemplates supplements made of capsules weighing between 200 mg to 500 mg, containing oxidized microparticles carriers, made of textile cellulose cotton gauze carriers, on the order between 0.675-97.2 μm. Its oxidation degree range between 0.208% w/w considerably 0.026 mg equiv. to 12.0% w/w considerably 1.5 mg-equiv. of aldehyde groups per gram of carrier, containing at least one bioactive substance or more bioactive substances of animal, flora origin or combination of bioactive substances and synthetic chemicals, having therapeutic activity. Some aspects include where the one bioactive substance is trypsin in various weights. Wherein the trypsin's dosages vary considerably from 50 mcg to 50 mg per capsule. Some aspects include where the one bioactive substance is insulin in various weights, wherein the insulin dosages vary considerably from 25 U of insulin per 1 g of capsules up to 1,000 U of insulin per 1 g of capsules. Some aspects include where the one bioactive substance is curcumin in various weights wherein the curcumin's dosages vary considerably from 100 mcg up to 25 mg per 1 capsule. Some aspects include where the one bioactive substance is resveratrol in various weights, wherein the resveratrol dosages vary considerably from 25 mcg to 15 mg per 1 capsule. Some aspects include where the one bioactive substance is cannabis in various weights and the dosages vary from 10 mg to 25 mg per 1 capsule. Some aspects include where the one bioactive substance is SOD in various weights and dosages. Some aspects include where the one bioactive ingredient is any protein in various weights and dosages. Some aspects include where the two bioactive substances are trypsin and insulin in various weights and dosages. Some aspects include where the two bioactive substances are trypsin and lysozyme in various dosages. Some aspects include where the two substances are Trypsin and resveratrol or resveratrol and curcumin. Some aspects include where the two or more bioactive ingredient are any protein or combination of proteins or compositions of proteins, synthetic chemicals and/or food ingredients in various dosages.

Some aspects of the invention include a dietary supplement mixture in the form powder containing net: said resveratrol microparticles carriers—0.50 g; Curcumin (extract) 1.29 g; Ginger 1.08 g; and Arabic coffee 2.13 g. Total unit weight 5 g packed in envelope/pouch. Some aspects include where the powder containing net: said Mexidole microparticles—0.50 g; Curcumin (extract)—1.29 g; Ginger—1.08 g; and Arabic coffee—2.13 g, Total unit weight 5 g. packed in envelope/pouch. Some aspects include where the powder containing net: 50 mg microparticles carriers, which comprise 5 mcg resveratrol; 25 mg microparticles which comprise 2.5 mcg trypsin; 35 mg microparticles which comprise 3.5 mcg curcumin; sugar 1,100 mg; lactose—365 mg; corn syrup solids—365 mg; carbon dioxide—247.5 mg; natural and artificial flavors—82.5 mg; caffein—80 mg; coconut oil—100 mg; malic acid—45 mg; zinc—2.5 mg. sucralose—2.5 mg. Total unit net weight 2.5 g, packed in an envelope/pouch. Some aspects include where the powder containing net: said resveratrol microparticles carriers—50 mg; isomaltose—1575 mg; xylitol—355 mg; carbon dioxide—175 mg; natural peppermint—140 mg; caffein—100 mg; coconut oil 92.5 mg; natural flavor—7.5 mg; zinc—2 mg. Total unit net weight 2.5 g. Each unit packed in envelope/pouch. Some aspects include where the powder containing net: Mexidole microparticles carriers—50 mg; zinc—2.5 mg; sugar—1125 mg; lactose—370 mg; corn syrup solid—370 mg; carbon dioxide—250 mg; caffein—80 mg; coconut oil 100 mg; natural flavor—82.5 mg; malic acid—45 mg; sucralose—25 mg. Total net weight 2.5 g, packed in envelope/pouch. Some aspects include where the powder containing net: trypsin and mexidole microparticles carriers—50 mg; isomaltose—1575 mg; xylitol—355 mg; carbon dioxide—175 mg; natural peppermint—140 mg; caffein—100 mg; coconut oil 92.5 mg; natural flavor—7.5 mg; zinc—5 mg. Total unit net weight 2.5 g, packed in envelope/pouch.

Some aspects of the invention include Bio-creams, Bio-Serums Bio-Shampoo containing oxidized cellulose microparticles carriers of cotton gauze or textile knitted caproamide fibers, having aldehyde groups between about 0.208% w/w considerably 0.026 mg equiv. to 12.0% w/w considerably 1.5 mg-equiv. per gram of carrier, containing at least one bioactive substance or more bioactive substances, of animal, flora origin or combination of bioactive substances and synthetic chemicals having therapeutic activity releasable properties. Some aspects include where the at least one bioactive substance is trypsin. The dosage of the trypsin varies from 10 mcg to 50 mcg per 99 g of cream or serum. Some aspects include where the at least one bioactive substance is lysozyme. Some aspects include where the at least one bioactive substance is resveratrol. Some aspects include where the at least one bioactive substance is mexidole. Some aspects include where the at least one bioactive substance is insulin. Some aspects include where the at least one bioactive substance is cannabis. Some aspects include where the at least one bioactive substance is superoxide dismutase. Some aspects include where the at least one bioactive substance is cannabis. Some aspects include where the at least one bioactive substance is chlorhexidine. Some aspects include where the microparticles carriers contains two bioactive substances. Some aspects include where the bioactive substance are trypsin and lysozyme. Some aspects include where the more than one bioactive substance is trypsin and insulin. Some aspects include where the micro-particles carriers contains bioactive substances. Some aspects include where the bioactive substance are trypsin, resveratrol and curcumin. Some aspects include where the bioactive substance are trypsin, resveratrol and lysozyme. Some aspects include where the bioactive substance is trypsin.

Some aspects include where the bioactive substance(s) is 0.03%

The materials and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, derivatives, formulations and/or methods of the invention may be made without departing from the spirit of the invention and the scope of the appended claims.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or subrange within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In the claims articles such as “a,”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

The following examples describe certain embodiments of the invention and and should not be construed as limiting the scope of what is encompassed by the invention in any way.

### EXAMPLES

### Example 1

### Cotton Fiber-Derived Microcellulose Carrier Conjugated Insulin and Trypsin Incorporated in Topical Formulations Promotes Accelerated Wound Healing

To incorporate insulin and trypsin on the same carrier, while preventing trypsin degradation of another therapeutic protein, a dressing comprising 10 mg trypsin and 20 U Insulin per 1 g was prepared and compared to other commercially available dressings: 1) Hospital Standard procedure 2) Dalcex Trypsin (“Trypsin”) dressing and 3) Trypsin & Insulin dressing. Table 1 depicts the results of this comparison study.

Treatment of 30 patients, suffered of Trophic Ulcers of the lower extremities. The patients were divided to 3 groups. Each group—10 patients, randomly chosen. Each group was treated with a different dressing material.

| | | | Granulation and | | Complete Healing | |
|---|---|---|---|---|---|---|
| | 2. Ulcer depuration | | epithelialization | | of the wounds | |
| | | Healing | | Healing | | Healing |
| 1. Treatment | Days in | acceleration | Days in | acceleration | Days in | acceleration |
| Dressing | average | % | average | % | average | % |
| Hospital Standard | 7.5 | Basis | 12 | Basis | 20.4 | Basis |
| Dalcex Trypsin | 2.4 | 68% | 4.5 | 62.5% | 15 | 26.5% |
| (50 mg/1 g) | | | | | | |
| Trypsin (50 mg) | 2.0 | 73% | 3.5 | 70.8% | 10.1 | 50.4% |
| & Insulin | | | | | | |
| (500 U per 1 g.) | | | | | | |

Surprisingly, dressings containing the conjugated trypsin—insulin combination was significantly more effective than a dressing comprising trypsin only or as compared to a hospital standard.

Sustained trypsin/insulin concentration within the dressing was also verified by Spectrophotometry. Concentrations remained the same throughout the duration of the test period.

### Example 2

Cotton Fiber-Derived Microcellulose Carrier Conjugated Insulin when Taken Orally is Effective

Eight (8) healthy rabbits of the New Zealand White variety, identified by ear tag, were obtained from a licensed supplier. Rabbits were individually housed in suspended cages and received commercially pelleted rabbit feed on a daily basis; tap water was freely available.

One Rabbit was orally administered respectively to the animal's weight with the Oral Solution made according to Russian patent RU (11)228000(13) C1. The Solution with dosage: ratio −0.7 U per one Kg of animal.

One Rabbit—got an APIDRA hypodermic Injection (made by Sanofi) at ratio dosage of 0.5 U per one Kg of animal, respectively to animal's weight.

3 Rabbits were orally administered, respectively to animals' weight, with Insulin tablets containing cotton microparticles carriers on the order of 0.675-97.2 μm with a dosage ratio: 0.5 U per one Kg of animal, made according to the present invention—marked T−1.

3 Rabbits were orally administered respectively to animals' weigh with Insulin Tablets containing cotton microparticles carriers on the order of 0.675-97.2 ?m, with a dosage ratio 0.5 U per one Kg of animal, made accorfing to the present invention—marked T −2.

The reaction, i.e., the glucose concentration in blood was measured from each rabbit ear on axis time of 30, 60, 90, and 120 minutes up to 24 hours.

The results of the test scoring appear in Table 1.

### Table 2:

A Comparison Test in using Insulin Drugs with Rabbits

| | | | Glucose concentration in Rabbits' blood, | | | | | | | | | |
|---|---|---|---|---|---|---|---|---|---|---|---|---|
| | | | mg/100 ml in N hours | | | | | | | | | |
| | Rabbit | IU | N hours | | | | | | | | | |
| | weight | per 1 | 0 | 0.5 | 1.0 | 1.5 | 2.0 | 4 | 8 | 20 | 22 | 24 |
| Description | Kg | Kg W. | Glucose concentration—mg/dl | | | | | | | | | |
| According to patent RU | 2.95 | 0.7 | 75 | 80 | 52 | 54 | 59 | 75 | 75 | 75 | 75 | 75 |
| (11)228000(13)C1 | | | | | | | | | | | | |
| Oral Solution % | | | | | | | | | | | | |
| hypodermic | 2.90 | 0.5 | 97 | 58 | 51 | 53 | 60 | 97 | 97 | 97 | 97 | 97 |
| INJECTION | | | | | | | | | | | | |
| Pills of microparticles | 3.05 | 0.5 | 100 | 100 | 100 | 100 | 85 | 70 | 55 | 55 | 70 | 80 |
| (T − 1) | 2.98 | 0.5 | 75 | 75 | 75 | 75 | 60 | 55 | 60 | 60 | 65 | 75 |
| | 2.85 | 0.5 | 100 | 100 | 100 | 100 | 85 | 70 | 55 | 58 | 70 | 100 |
| Pills of microparticles | 3.00 | 0.5 | 103 | 103 | 103 | 103 | 60 | 50 | 40 | 40 | 60 | 80 |
| (T − 2) | 2.95 | 0.5 | 85 | 85 | 85 | 85 | 50 | 45 | 45 | 45 | 50 | 60 |
| | 2.97 | 0.5 | 95 | 95 | 95 | 95 | 60 | 50 | 40 | 45 | 55 | 75 |

### Results:

One Rabbit that was orally administered respectively to the animal's weight with the oral solution made according to the Russian patent RU (11)228000(13) C1. The dosage was calculated based on 0.7 IU per one kg of the animal weight The Solution was effective during the first 2 hours. After which glucose climbed back to initial levels. The main drawback of the specific method is a short-time hyperglycemic effect.

One Rabbit which got an APIDRA hypodermic Injection made by Sanofi with dosage ratio—5.0 U per one Kg of animal injected respectively to animal's weight. The hypoglycemic effect. was as expected effective for 2 hours.

3 Rabbits—were administered with insulin tablets of microparticles carriers made according to the present invention (marked: T−1), which were orally administered respectively 0.5 U per one kg of animals' weight passed the stomach, withstands the gastric juice and pressure and was effective during 22-24 hours.

3 Rabbits—were administered with insulin tablets of microparticles carriers made according to the present invention (marked: T−2), which were orally administered respectively 0.5 U per one kg of animals' weight passed the stomach, withstands the gastric juice and pressure was effective during 24 hours.

The Oral solution as per the Russian Patent RU (11) 228000(13)C1 was effective as expected.

The Hypodermic injection APIDRA was effective as expected.

The insulin molecules within the insulin tablets made according to the present invention averted degradation by the digestive enzymes as evidenced by the reduced circulating glucose level.

Microparticles carriers' lengths in a given volume follow a Gaussian distribution. Outwardly, this material resembles microcrystalline cellulose (MCC), which is used as the inert part of medicinal tablets, dietary supplements and cosmetic mixtures and also as a filler and special additive for some technical articles. It is known that the size of microcrystalline cellulose is in the range of 30-50 μm, and the method for obtaining it—cellulose de-polymerization in solutions of mineral acids—excludes the possibility of obtaining from MCC therapeutic materials possessing directed medicinal activity. Therefore, in processes for obtaining therapeutic materials, MCC is used as inert filler.

It is also known that the microcrystalline part of cotton cellulose (CC) is about 70% and the amorphous part, about 30% by weight. In this way, 30% of the CC is converted into production wastes and together with mineral acids and other additives requires building expensive water treatment facilities.

Resulting of the Vortex grinding—milling process the therapeutical properties are also preserved within the amorphous part and it is also could be used with no additives or any additional treatment as an active component of the tablet(s)

Well known in the art, that covalent bonding of proteins to DAC converts them to an insoluble state, which leads to a reduction in their specific activity. The covalent bonding of proteins, e.g., trypsin, lysozyme, insulin and/or others to DAC, due to the formation of an azomethine bond between the aldehyde groups of the DAC and the amino groups of the protein side chains, increases their stability with respect to inactivating agents: heat, inhibitors, proteolytic agents and ionizing radiation. (See, for example, V.N. Filatov, V.V. Ryltsev. Bioactive textile materials. M: Informelektro, 2002. Pages 147-165.)

Furthermore, the cellulose microparticles carriers, made according to present invention, which composing the tablets and the capsules containing dialdehyde groups (DAC), and comprising bio-active substances have in contrast to cellulose, an unusual property.

DAC carriers are capable of hydrolytic degradation in an aqueous environment. In this case, small fragments of DAC (molecules, dimmers and so forth), with the proteins immobilized on them, go into gastric juice solution. Unexpectedly and surprisingly such low-molecular protein-DAC conjugates possess greater specific activity than high-molecular conjugates and greater stability in solution than native proteins. Whereas the hydrolytic degradation rate constant depends greatly on the pH of the environment. In a very acidic environment, breakdown is practically not observed. In a neutral environment, it is 18.0×10-5 min-1. In pH=8, it is 13.8×10-4. But in pH=9.2, it is 42.1×10-4 min-1, i.e., an increase of pH to the alkaline side of 1 unit from neutral leads to an increase in the breakdown speed by 1 order; an increase of 2 units, by more than 2 orders. (See, for example, V.N. Filatov, V.V. Ryltsev. Bioactive textile materials. M: Informelektro, 2002. Page 89.) Since the pH of gastric juice is 0.8-1.5 and that of the small intestine is 7.2-8.6, DAC carriers as according to the present invention can be considered an optimum carrier of medicines containing free amino groups in their composition. In the stomach, the DAC-insulin conjugate is protected from the destructive action of the environment; in the small intestine, dissoluble fragments are washed out of it, which diffuse into the blood, producing a hypoglycemic effect.

Cotton fibers have a naturally a spiral property. Because of the Vortex grinding—milling process, the spiral properties and the bio-active substances' properties which are bound within the fibrous carrier are preserved. Once these microparticles carriers are being transformed to tablets or capsules these properties seemingly continue to be preserved within the tablets and the capsules.

In the aggressive environment of the stomach, the microparticles carriers within the tablets are being disintegrated. However, because of its tiny sizes the microparticles acquire a globular form, which provides additional protection to the bio-active substance (s) immobilized thereon from breaking down and inactivation.

In the small intestine, the globule unfolds and under the influence of hydrolytic degradation breaks down into small fragments containing insulin, which are absorbed into the blood.

The present invention Inter alia provides a method to create a protection shell and releasable properties by providing the carriers the following properties:

Dialdehyde Groups—respectively, and equally spread.

Adjusted pH

Microparticles carriers having spiral properties that enable the carriers to acquire a globular form

At least one Bio-Active Substance or a plurality of Bio-Active substances; about 20% which are covalently bound and about 80% bound by sorption.

Consequently, proteins having the necessary protection to survive the digestive enzymes' attack and the pressure in the gastrointestinal track, passing hydrolysis processes and keeping its therapeutical properties, entering the blood system through the small intestine wall, releasing its therapeutical properties effectively as 95%-97% of the therapeutical agent is being released fast and effective as native species and doing the job.

From the aforesaid, it will be understood that since macromolecules such as insulin passed the gastrointestinal track and did the job successfully other molecules, especially relatively smaller molecules (like Trypsin) can successfully pass the gastrointestinal tract intact, released, enter the bloodstream, and do the job as designed effectively.

### Example 3

Trypsin tablets containing cellulose microparticles carriers that varies between 0.675-97.2 μm, partially oxidized, comprising the bioactive substance were made as follow: medical cotton gauze was activated by oxidation with periodate nitrate to prepare dialdehyde cellulose carrier as follows:

8 L of water were poured into a reactor, mixed with a stirrer and 30 g of periodate nitrate were added thereto. The periodate nitrate was dissolved within 5-10 minutes. The resulted solution has a pH of 5.0.

1 Kg of medical cotton gauze was placed into the resulting solution and held (activated) at room temperature for 14 hours in the dark. After activating, the cloth was squeezed out, washed with 4×8 L of water, squeezed again and dried.

As a result of activation, the cotton cloth acquires 1.0% w/w of aldehyde groups corresponding to 0.125 mg-equiv. per gram of the textile carrier.

A bio-active solution—a solution containing trypsin is prepared in phosphate buffer having a pH of 5.5 as follows: 10 g of trypsin were added to 4.2 of the prepared phosphate buffer solution, stirring until it was completely dissolved. The activated textile carrier was reacted by impregnation during 2 hours in the dark. The resulting material was held in air and dried until residual moisture is no more than 10% at a room temperature. The resulted cotton cloth contains 0.01 g of trypsin per 1 gram of the carrier.

Then, by a Vortex milling machine 1 kg of the obtained cloth was grinded—milled to microparticles particles in the form of a dry powder, on the order of 0.675-97.2 μm. Properties of said treated cloth were entirely preserved so the DAC microparticles carriers are comprising 0.01 g of trypsin per 1 g of microparticles carriers, i.e., the trypsin dosage is 1.0%

Trypsin tablets weighing 200 mg (each) were made from said microparticles carriers of 0.675-97.2 μm, having 0.125 mg-equiv. (1%) dialdehyde groups, comprising 1% trypsin, by a Ronchi tablets press machine or could be done by a similar pressing machine. Each of the resulted tablets contain 2 mg of trypsin per 1 tablet.

### Example 4

Trypsin tablets containing cellulose microparticles carriers weighing 200 mg. were made following Example 1 except that oxidation degree of said microparticles carriers is 0.375 mg-equiv. and the amount of trypsin added to the trypsin solution is 20 g.

Further treatment was carried out following the procedure of Example 1 and each of the resulted tablets contains 4 mg trypsin.

### Example 5

Trypsin tablets containing cellulose microparticles carriers at the size of 0.675-97.2 μm, partially oxidized, comprising the bioactive substance weighing 200 mg. were made as follow: medical cotton gauze was activated to obtain dialdehyde cellulose following the procedure of Example 1, except that to the reactor which contained 8 L of water 185 g of periodate nitrate were added thereto and stirred. The periodate nitrate was dissolved within 5-10 minutes. The resulted solution has a pH of 5.0

1 Kg of medical cotton gauze was placed into the resulting solution and held (activated) at room temperature for 24 hours in the dark. After activating, the cloth was squeezed out, washed with 4×10 L of water, squeezed again and dried.

As a result of activation, the cotton cloth acquires 6.0% w/w of aldehyde groups corresponding to 0.75 mg-equiv. per gram of the textile carrier.

Further treatment was carried out following the procedure of Example 1 except that 50 g of trypsin were added to 4.2 L of the prepared phosphate buffer solution (trypsin solution) having a pH of 5.5. Consequently, the resulting carriers all along the way including the microparticles carriers contain 5% w/w of trypsin per 1 g of carrier. and,

Further treatment was carried out following the procedure of Example 1.

The resulted tablets of 200 mg each contains 10 mg trypsin per tablet.

### Example 6

Trypsin tablets containing cellulose microparticles carriers at the size of 0.675-97.2 μm, partially oxidized, comprising the bioactive substance weighing 200 mg were made as follow: medical cotton gauze was activated to obtain dialdehyde cellulose following the procedure of Example 1, except that to the reactor which contained 8 L of water 372 g of periodate nitrate were added and thereto and stirred. The periodate nitrate was dissolved within 5-10 minutes. The resulted solution has a pH of 5.0

1 Kg of medical cotton gauze was placed into the resulting solution and held (activated) at room temperature for 24 hours in the dark. After activating, the cloth was squeezed out, washed with 4×10 L of water, squeezed again and dried.

As a result of activation, the cotton cloth acquires 12.0% w/w of aldehyde groups corresponding to 1.5 mg-equiv. per gram of the textile carrier.

Further treatment was carried out following the procedure of Example 1 except that 100 g of trypsin were added to 4.2 L of the prepared phosphate buffer solution (trypsin solution) having a pH of 5.5. Consequently, the resulting carriers all along the way including the microparticles carriers contain 10% w/w of trypsin per 1 g of Carrier. Consequently, the resulted tablets of 200 mg each contains 20 mg trypsin per tablet.

### Example 7

Trypsin tablets containing cellulose microparticles carriers weighing 250 mg. were made following the procedure of Example 3 except that the oxidation degree of said microparticles carriers is 1.5 mg. equiv. and the (added) amount of trypsin for preparing the trypsin solution is 300 g.

Further treatment was carried out following the procedure of Example 1, except that each tablet weighing 250 mg contains 75 mg trypsin.

### Example 8

Trypsin tablets containing cellulose microparticles carriers weighing 1,000 mg. were made following Example 3 except that the oxidation degree of said microfibrill carriers is 1.5 mg. equiv. and the (added) amount of trypsin for preparing the trypsin solution is 300 g.

Further treatment was carried out following the procedure of Example 1 and each of the resulted tablets contains 300 mg trypsin.

### Example 9—Insulin

Insulin tablets containing cellulose microparticles carriers on the order of 0.675-97.2 μm, partially oxidized, comprising the bioactive substance were made as follow: medical cotton gauze was activated to obtain dialdehyde cellulose following the procedure of Example 1, except that to the reactor which contained 8 L of water, 185 g of periodate nitrate were added thereto and stirred. The periodate nitrate was dissolved within 5-10 minutes. The resulted solution has a pH of 5.0

1 Kg of medical cotton gauze was placed into the resulting solution and held (activated) at room temperature for 14 hours in the dark. After activating, the cloth was squeezed out, washed with 4×10 L of water, squeezed again and dried.

As a result of activation, the cotton cloth acquires 6.0% w/w of aldehyde groups corresponding to 0.75 mg-equiv. per gram of the textile carrier.

A solution of insulin is prepared in phosphate buffer having a pH of 5.5 as follows: 3,627 g of insulin were added to 3.3 L of said phosphate buffer solution, stirred until it was completely dissolved. Then 1 kg of medical cotton gauze was activated by impregnation during 2 hours in the dark. The resulting material was held in air and dried until residual moisture is phosphate buffer solution of pH 5.5 is no more than 10% at a room temperature. The resulted cotton cloth 1 kg contains 3,627 g insulin corresponding to 100,000 U, i.e. 3.637 mg per 1 gram of carrier corresponding to 100 U insulin per 1-gram cloth-carrier.

The obtained material was then grinded-milled by a Vortex milling machine and was transformed into microparticles carriers on the order of 0.675-97.2 μm. The resulting microparticles carriers contain 3.637 mg per 1 gram of carrier corresponding to 100 U insulin per 1-gram of microparticles carrier.

Then, insulin tablets weighing 100 mg containing said microparticles carriers on the order between 0.675-97.2 μm were made by a Ronchi tablets press machine and/or could be done a similar press machine. Each of the 100 mg tablets, which made of said microparticles carriers contains 10 U insulin.

### Example 10

Insulin tablets containing cellulose microparticles carriers were made following the procedure of example 6 except that for preparing the insulin solution 18,185 mg of insulin were added to 4.2 L of the phosphate buffer solution having a pH of 5.5, and, that the resulted dry cotton cloth was grinded—milled to microparticles on the order of 0.675-97.2 μm, which contains 18,135 mg corresponding to 500,000 U per 1 Kg of cloth-carrier. i.e., 500 u per 1 g of carrier.

Further treatment of producing tablets was carried out following the procedure of Example 6 except that each tablet which is 100 mg comprising 50 U insulin.

### Example 11

Insulin tablets containing microparticles carriers were made following the procedure of example 6 except that for preparing the insulin solution 21,762 mg of insulin were added to 4.2 L of the phosphate buffer solution having a pH of 5.5, and, that the resulted dry cotton cloth was grinded—milled to microparticles on the order of 0.675-97.2 μm. The resulted microparticles carriers contain 21,762 mg insulin corresponding to 600,000 U per 1 Kg of cloth-carrier.

Further treatment of producing tablets was carried out following the procedure of Example 7 except that each tablet weighing 200 mg and comprising 120 U insulin.

### Example 12

Insulin tablets containing microparticles carriers, weighing 250 mg each, were made following the procedure of example 7 except that 40,000 units of insulin solution (40 vails, each contains 1,000 U, in total 40,000 U) were added to 4.2 L of the phosphate buffer solution having a pH of 5.5, and, that the resulted dry cotton cloth was grinded—milled to microparticles particles on the order of 0.675-97.2 μm. The resulted microparticles carriers contain 40,000 U insulin per 1 kg carrier corresponding to 40 U insulin per 1 g of cloth-carrier.

Further treatment of producing tablets was carried out following the procedure of the procedure of Example 7 except that each tablet which is 250 mg comprising 10 U insulin.

### Example 13

Tablets containing insulin and trypsin weighing 100 mg (each) were made following the procedure of example 7 except that the oxidation degree of the cloth-carrier is 6% corresponding to 0.75 meg-equiv., and for preparing the bio-active insulin solution, 18,135 mg of insulin per 1 kg of cotton cloth was added to 4.2 L of the phosphate buffer solution having a pH of 5.5, and for preparing the trypsin, solution following the procedure of Example 3, 10 g of trypsin per 1 kg of cotton cloth were put into a separate reactor containing 4.2 L of buffer solution having pH of 5.5. The insulin solution and the trypsin solution were mixed at the ratio of 1/1 and agitated during 5-7 min.

Further treatment was carried out following the procedure of Example 1. except that the cotton cloth was grinded—milled to microparticles carriers on the order of 0.675-97.2 μm. Each tablet of 100 mg comprising 50 U insulin and 10 mg trypsin.

### Example 14

Tablets containing insulin and trypsin weighing 100 mg (each) were made following the procedure of example 7 except that the oxidation degree of the cloth-carrier is 6% corresponding to 0.75 meg-equiv., and for preparing the bio-active insulin solution, 21,762 mg of insulin per 1 kg of cotton cloth was added to 4.2 L of the phosphate buffer solution having a pH of 5.5, and 10 g of trypsin per 1 kg of cotton cloth were put into the bio-active trypsin solution that was prepared the bio-active trypsin solution was prepared following the procedure of Example 3. The insulin solution and the trypsin solution were mixed at the ratio of 1/1 and agitated during 5-7 min.

Further treatment was carried out following the procedure of Example 1. except that the cotton cloth was grinded—milled to microparticles carriers on the order of 0.675-97.2 μm. Each tablet of 100 mg comprising 60 U insulin and 20 mg trypsin.

### Example 15

Tablets weighing 250 mg (each) containing insulin and trypsin were made following the procedure of example 7 except that for preparing the bio-active insulin solution, 14,508 mg of insulin per 1 kg of cotton cloth was added to 4.2 L of a separate phosphate buffer solution having a pH of 5.5; and 10 g of trypsin per 1 kg of cotton cloth were put into the bio-active trypsin solution that was prepared following the procedure of Example 3. The insulin solution and the trypsin solution were mixed at the ratio of 1/1 and agitated during 5-7 min. The resulted carrier cloth contains 400,000 insulin units and 10 mg of trypsin per 1 kg. i.e., 400 insulin units and 10 mg per 1 g of carrier cloth.

Further treatment was carried out following the procedure of Example 1. except that the cotton cloth was grinded—milled to microparticles carriers on the order of 0.675-97.2 μm. Each tablet of 250 mg comprising 100 U insulin and 10 mg trypsin.

### Example 16

Tablets containing trypsin and lysozyme weighing 200 mg (each) were made following the procedure of example 11 except that additional bio-active lysozyme solution was also prepared as follows: 0.5 g of lysozyme was added to 3.3 L of the phosphate buffer solution having a pH of 5.5, and stirred. trypsin solution was prepared following the procedure of Example 3. The trypsin solution and the lysozyme solution were mixed at the ratio of 1/1 and agitated during 5-7 min.

Further treatment was carried out following the procedure of Example 3. Each tablet (of 200 mg) comprising 10 mg trypsin and 0.5 mg of lysozyme.

### Example 17

Tablets of said cellulose particles carriers weighing 250 mg. containing glatiramer acetate (copaxsone) were made following Example 3 except that 40 mg/ml×4,000 units of glatiramer acetate (copaxsone) were added to the 4.2 L bioactive solution and stirred 10 min.

Further treatment was carried out following the procedure of Example 1. Each of the resulted tablets contains 40 mg of glatiramer acetate (copaxsone).

### Example 18

Tablets of said cellulose microparticles carriers weighing 200 mg. containing glatiramer acetate (copaxsone) were made following Example 3 except that 20 mg/ml×5,000 units of glatiramer acetate (copaxsone) were added to the 4.2 L bioactive solution and stirred 10 min.

Further treatment was carried out following the procedure of Example 1. Each of the resulted tablets contains 20 mg of glatiramer acetate (copaxsone).

### Example 19

Tablets of said cellulose microparticles carriers weighing 250 mg. containing chitosan and comprising glatiramer acetate (copaxsone) were made following Example 3 except that 1 g Chitosan was added to 50 ml N-methyl-2-pyrrolidinone and treated with 15 ml of 1.5 N NaOH solution. The mixture was stirred for 30 min. at 50° C.; and were then added to 3.8 L of phosphate buffer solution at 5.5 pH, which was then stirred for 2 hours at 50° C. The mixture was then cooled.

40 mg/ml×4,000 units of glatiramer acetate (copaxsone) were added to the obtained mixture solution and stirred 10 min.

Further treatment was carried out following the procedure of Example 1. Each of the resulted tablets contains 40 mg of glatiramer acetate (copaxsone).

### Example 20

Tablets of said cellulose microparticles carriers weighing 250 mg. containing chitosan and comprising glatiramer acetate (copaxsone) were made following Example 3 except that 1 g Chitosan was added to 50 ml N-methyl-2-pyrrolidinone and treated with 15 ml of 1.5 N NaOH solution. The mixture was stirred for 30 min at 50° C. and were then added to 3.8 L of phosphate buffer solution at 5.5 pH, which was then stirred for 2 hours at 50° C. The mixture was then cooled.

20 mg/ml×4,000 units of glatiramer acetate (copaxsone) were added to the obtained mixture solution and stirred 10 min.

Further treatment was carried out following the procedure of Example 1. Each of the resulted tablets contains 20 mg of glatiramer acetate (copaxsone).

### Example 21

Tablets containing resveratrol and curcumin were made following the procedure of Example 11 except that 40 g resveratrol were added to one of phosphate buffer solution of pH 5.5, and 40 g of curcumin were added to the second phosphate buffer solution.

Further treatment was carried out following the procedure of Example 11. Each of the resulted tablets weighing 250 mg contains 10 mg of resveratrol and 10 mg of curcumin.

### Example 22

Tablets containing cannabis were made following the procedure of Example 3, except that 50 g of CBD or THC, alcohol extracted, at purity degree of 85%-95%, was added to 300 ml of ethanol and added to 3.0 L of a prepared phosphate buffer solution having a pH of 5.5. The temperature was adjusted to 60° C. and the obtained bio-active solution was stirred and boiled during 15 min, and then cooled.

1 Kg of medical cotton gauze was placed into the resulting solution and held (activated) at room temperature for 24 hours in the dark. After activating, the cloth was dried

Further treatment was carried out following the procedure of Example 1, except that each tablet weighing 200 mg contains 10 mg of CBD or THC.

### Example 23

Tablets containing cannabis were made following the procedure of Example 3, except that 125 g of CBD or THC, alcohol extracted at purity degree of 85%-95% was added to 300 ml of ethanol and added to 3.0 L of a prepared phosphate buffer solution having a pH of 5.5. The temperature was adjusted to 60° C. and the obtained bio-active solution was stirred and boiled during 15 min, and then cooled.

1 Kg of medical cotton gauze was placed into the resulting solution and held (activated) at room temperature for 24 hours in the dark. After activating, the cloth was dried

Further treatment was carried out following the procedure of Example 1, except that each tablet weighing 200 mg contains 25 mg of CBD or THC.

### Example 24

Resveratrol capsules containing cellulose microparticles carriers were prepared following Example 3, except that 50 g of resveratrol was added to the phosphate buffer solution. Further treatment was carried out following the procedure of Example 1, except that the treated cloth was milled to microparticles carriers on the order of 0.675-97.2 μm (in the form of dry powder). Said microparticles carriers were used to fill capsules. Each capsule weighing 200 mg contains 10 mg of resveratrol.

### Example 25

Resveratrol capsules containing cellulose microparticles carriers were prepared following Example 13, except that to the phosphate buffer solution 100 g of resveratrol was added. Further treatment was carried out following the procedure of Example 1, except that Each capsule is weighing 250 mg contains 25 mg of resveratrol.

### Example 26

Curcumin capsules containing cellulose microparticles carriers were prepared following Example 3, except that 200 g of curcumin was added to the phosphate buffer solution. Further treatment was carried out following the procedure of Example 1. Said microparticles carriers in the form of day powder were used to fill capsules. Each capsule which is weighing 200 mg contains 40 mg of curcumin.

### Example 27

Curcumin capsules containing cellulose microparticles carriers were prepared following Example 19, except that 400 g of curcumin was added to the phosphate buffer solution. Further treatment was carried out following the procedure of Example 17, except that Each capsule weighing 250 mg containing 100 mg of curcumin.

### Example 28

Capsules containing cannabis were made following the procedure of Example 3, except that 50 g of CBD or THC, alcohol extracted, at purity degree of 85%-95%, was added to 300 ml of ethanol and added to 3.0 L of a prepared phosphate buffer solution having a pH of 5.5. The temperature was adjusted to 60° C. and the obtained bio-active solution was stirred and boiled during 15 min, and then cooled.

1 Kg of medical cotton gauze was placed into the resulting solution and held (activated) at room temperature for 24 hours in the dark. After activating, the cloth was dried

Further treatment was carried out following the procedure of Example 1, except that each capsule weighing 200 mg contains 10 mg of CBD or THC.

### Example 29

Capsules containing cannabis were made following the procedure of Example 3, except that 125 g of CBD or THC, alcohol extracted at purity degree of 85%-95% was added to 300 ml of ethanol and added to 3.0 L of a prepared phosphate buffer solution having a pH of 5.5. The temperature was adjusted to 60° C. and the obtained bio-active solution was stirred and boiled during 15 min, and then cooled.

1 Kg of medical cotton gauze was placed into the resulting solution and held (activated) at room temperature for 24 hours in the dark. After activating, the cloth was dried

Further treatment was carried out following the procedure of Example 1, except that each capsule weighing 200 mg contains 25 mg of CBD or THC.

### Example 30

Dietary supplement in the form of dry powder containing a mix of resveratrol, curcumin, ginger and Arabic coffee, packed in envelopes (units) net weight per unit 5 g, was made as follow:

Cellulose microparticles carriers of 0.675-97.2 μm comprising 15% resveratrol was made following Example 3 except that 150 g of resveratrol were added to bio-active—resveratrol solution. The further treatment was made following example 3.

As to get 1 Kg of a dietary supplement the obtained microparticles carries were mixed with the following components as follows:

Said resveratrol microparticles carriers—100 g. (10%). Curcumin (extract) 258 g. (25.8%). Ginger—216 g. (21.6%) and Arabic coffee 426 g. (42.6%)—total 1 kg

The resulted mixture was packed in envelopes. Each envelope (unit) net weighing 5 g. containing: said resveratrol microparticles carriers—0.50 g; Curcumin (extract) 1.29 g; Ginger 1.08 g; and Arabic coffee 2.13 g.

### Example 31

Dietary supplement in the form of dry powder containing a mix of mexidole, curcumin, ginger and Arabic coffee, net weight per unit 5 g, packed in envelopes was made following Example 1 except that 150 g of mexidole was added to the phosphate buffer solution. Further treatment was made following the procedure of Example 25, except that the resulted mixture was packed in envelopes, each envelope/unit weighing 5 g. contains (net): said microparticles containing Mexidole—0.50 g; Curcumin (extract)—1.29 g; Ginger—1.08 g; and Arabic coffee—2.13 g.

### Example 32

Dietary supplements mixture in the form of dry powder containing resveratrol, trypsin, curcumin, sugar, lactose, corn syrup solid, carbon dioxide, caffein, coconut oil, natural flavor, malic acid, sucralose and zinc, packed in pouches were made as follow:

Cellulose microparticles carriers of 0.675-97.2 μm comprising resveratrol was made following the procedure of Example 3 except that to each of the 3 separate buffer solution at 5.5 pH were added bio-active substances as follows: one—10 g of resveratrol; to the second—10 g of trypsin; to the third—10 g of curcumin. The further treatment was made separately per each of the cloth following the procedure of Example 3.

1 Kg of the dietary supplements made of a mixture of the following components was prepared as follows:

sugar 444 g; lactose—146 g; corn syrup solids—146 g; carbon dioxide—99 g; natural and artificial flavors—33 g; caffein—32 g; coconut oil—40 g; malic acid—18 g; zinc—1 g; and sucralose—1 g were poured into a reactor and stirred during 7—minutes. Then dried in an oven at 35° C.

Then, 20 g of resveratrol microparticles carriers and 10 g of trypsin microparticles and 14 g of curcumin microparticles were added to the mixture and stirred during 5 min.

The resulted mixture was packed in pouches. Each pouch/unit which is weighing 2.5 g. contains (net): 50 mg microparticles carriers, which comprise 5 mcg resveratrol; 25 mg microparticles which comprise 2.5 mcg trypsin; 35 mg microparticles which comprise 3.5 mcg curcumin; sugar 1,100 mg; lactose—365 mg; corn syrup solids—365 mg; carbon dioxide—247.5 mg; natural and artificial flavors—82.5 mg; caffein—80 mg; coconut oil—100 mg; malic acid—45 mg; zinc—2.5 mg. sucralose—2.5 mg

### Example 33

A dietary supplement mixture in the form of dry powder containing resveratrol, isomaltose, xylitol, carbon dioxide, natural peppermint flavor, caffeine, coconut oil and natural flavor, zinc, was made as follow:

Cellulose microparticles carriers of 0.675-97.2 μm comprising resveratrol was made following the procedure of Example 3 except that 20 g of resveratrol were added to phosphate buffer solution at 5.5 pH. The further treatment was made following the procedure of Example 27.

To prepare 1 Kg of the dietary supplements a mixture of the following components was made as follows:

Said resveratrol Microparticles carriers—20 g; isomaltose—630 g; xylitol—142 g; carbon dioxide—70 g; natural peppermint flavor—56 g; caffein—40 g; coconut oil 37 g; natural flavor—3 g; zinc—2 g.

The resulted mixture was packed in envelopes. Each envelope/unit which is weighing 2.5 g. contains (net):

Resveratrol microparticles carriers—50 mg; isomaltose—1575 mg; xylitol—355 mg; carbon dioxide—175 mg; natural peppermint—140 mg; caffein—100 mg; coconut oil 92.5 mg; natural flavor—7.5 mg; zinc—2 mg.

### Example 34

A dietary supplement mixture in the form of powder containing mexidole, zinc, sugar, lactose, corn syrup solid, carbon dioxide, caffein, coconut oil, natural flavor, malic acid, sucralose was made as follow:

Microparticles carriers comprising resveratrol was made following the procedure of Example 3 except that 20 g of mexidole were added to phosphate buffer solution at 5.5 pH. Further treatment was made following the procedure of Example 25.

To prepare 1 Kg of the dietary supplements a mixture of the following components was made as follows:

Said mexidole microparticles carriers—20 g; zinc—1 g; sugar—450 g; lactose—148 g; corn syrup solid—148 g; carbon dioxide—100 g; caffein—32 g; coconut oil 40 g; natural flavor—33 g; malic acid—18 g; sucralose—10 g.

The resulted mixture was packed in envelopes. Each envelope/unit which is weighing 2.5 g. contains (net):
Mexidole microparticles carriers—50 mg; zinc—2.5 mg; sugar—1125 mg; lactose—370 mg; corn syrup solid—370 mg; carbon dioxide—250 mg; caffein—80 mg; coconut oil 100 mg; natural flavor—82.5 mg; malic acid—45 mg; sucralose—25 mg.

### Example 35

A dietary supplement mixture in the form of powder containing trypsin, isomaltose, xylitol, carbon dioxide, natural peppermint flavor, caffeine, coconut oil and natural flavor, zinc, was made as follow:

Cellulose microparticles carriers of 0.675-97.2 μm comprising trypsin was made following the procedure of Example 3 except that 10 g of trypsin was added to phosphate buffer solution at 5.5 pH. and 10 g of mexidole was added to the second phosphate buffer solution a 5.5 pH. The two solution were mixed at the ratio of 1/1 and agitated during 5-7 min.

The further treatment was made following the procedure of Example 25.

To prepare 1 Kg of the dietary supplements a mixture of the following components was made as follows:

Said trypsin and mexidole microparticles carriers—20 g; isomaltose—630 g; xylitol—142 g; carbon dioxide—70 g; natural peppermint flavor—56 g; caffein—40 g; coconut oil 37 g; natural flavor—3 g; zinc—2 g.

The resulted mixture was packed in envelopes. Each envelope/unit which is weighing 2.5 g. contains (net):

trypsin and mexidole microparticles carriers—50 mg; isomaltose—1575 mg; xylitol—355 mg; carbon dioxide—175 mg; natural peppermint—140 mg; caffein—100 mg; coconut oil 92.5 mg; natural flavor—7.5 mg; zinc—5 mg.

### Example 36

Bio-active cream containing cellulose microparticles carriers on the order of 0.675-97.2 μm (in the form of dry powder) partially oxidized, comprising a bioactive substance—trypsin—was made as follow: medical cotton gauze was activated to obtain dialdehyde cellulose 30 g of periodate nitrate were added to the reactor which contained 8 L of water and stirred. The periodate nitrate was dissolved during 5-10 minutes. The resulted solution has a pH of 5.0

1 Kg of medical cotton gauze was placed into the resulting solution and held (activated) at room temperature for 14 hours in the dark. After activating, the cloth was squeezed out, washed with 4×10 L of water, squeezed again and dried.

As a result of activation, the cotton cloth acquires 1.0% w/w of aldehyde groups corresponding to 0.125 mg-equiv. per gram of the textile carrier.

Further treatment was carried out following the procedure of Example 1 except that to prepare a trypsin solution, 10 g of trypsin were added to 3.3 L contains phosphate buffer solution having a pH of 5.5. Consequently, the resulting carriers all along the way contain 1.0% w/w of trypsin per Carrier.

Then, by a Vortex milling machine 1 kg of the obtained cloth was grinded—milled to microparticles carries, in the form of dry powder, on the order of 0.675-97.2 μm following Example 1. The properties of said treated cloth were entirely preserved so the DAC microparticles carriers contains 10 mg of trypsin per 1 g of microparticles carriers.

One (1) g. of said bio-active microparticles carriers was add to 99 g of cream (any cream,) mixed 1-2 min by a mixer. The obtained cream contains 10 mg trypsin per 100 g cream

### Example 37

A bio-cream comprising trypsin was made following the procedure of Example 34 except that to the reactor which contained 8 L of water 90 g of periodate nitrate were added thereto and stirred. The periodate nitrate was dissolved within 5-10 minutes.

1 Kg of medical cotton gauze was placed into the resulting solution and held (activated) at room temperature for 14 hours in the dark. After activating, the cloth was squeezed out, washed with 4×10 L of water, squeezed again and dried.

As a result of activation, the cotton cloth acquires 3.0% of dialdehyde groups corresponding to 0.375 mg-equiv. per gram of the textile carrier.

Further treatment was carried out following the procedure of Example 36 except that 30 g of trypsin were added to bio-active—trypsin solution. The obtained cream contains 30 mg of trypsin per 100 g of cream.

### Example 38

A bio-cream comprising trypsin was made following the procedure of Example 34 except that to the reactor which contained 8 L of water 372 g of periodate nitrate were added and as a result of the activation the cotton cloth acquires 12% of dialdehyde groups corresponding to 1.5 mg-equiv. per one gram of textile carrier

Further treatment was carried out following the procedure of Example 36 except that 100 g of trypsin were added to the bio-active—trypsin solution. Subsequently the obtained cream contains 100 mg of trypsin per 100 g of cream.

### Example 39

A bio-cream comprising superoxide dismutase (SOD) was made following the procedure of Example 34 except that to the reactor which contained 8 L of water 185 g of periodate nitrate were added and as a result of the activation the cotton cloth acquires 6% of dialdehyde groups corresponding to 0.75 mg-equiv. per one gram of textile carrier

Further treatment was carried out following the procedure of Example—1 except that 50 g of SOD were added to the phosphate buffer solution.

Further treatment was carried out following Example 36. The obtained cream contains 50 mg of SOD per 100 g of cream.

### Example 40

Bio-cream containing cellulose microparticles carriers at the size of 0.675-97.2 μm partially oxidized comprising resveratrol was carried out following the procedure of Example 34, except that 185 g of periodate nitrate were added to the reactor which contained 8 L of water and stirred. Subsequently the obtain dialdehyde cellulose contain 0.75 mg-equiv. aldehyde groups corresponding to 6% w/w per gram of a carrier.

A solution of resveratrol was prepared in phosphate buffer solution following the procedure of Example 34 except that 50 g of resveratrol was added to 3.3 L phosphate buffer solution at pH 5.5, and the resulting mixture was stirred until completely dissolved.

1.0 Kg of the obtained oxidized textile carrier was immersed in the resulting solution for activation. The carrier was then kept 2 hours at room temperature, and then dried by air.

The further treatment was carried out following the procedure of Example 36. Subsequently the resulting cream contains 50 mg of resveratrol per 100 g of cream.

### Example 41

Bio-cream containing trypsin and resveratrol was made as follows: Medical cotton gauze was activated to obtain oxidized cellulose cloth containing 0.125 mg-equiv. of aldehyde groups corresponding to 1% (w/w) per gram of carrier, by following procedure Example 1. except that 50 g of 3-hydroxy-6-methyl-2-ethypyridine succinate (mexidole) was added to 3.3 L phosphate buffer solution at pH 5.5, and the resulting mixture was stirred to complete dissolution. 1 kg of the obtained oxidized cloth was placed into the mexidole solution for 2 hours at room temperature and then squeezed by a wringer between two rollers at a pressure of 25 Kg per 1 sq. cm and, dried by utilizing hot oven 90° C., or by forced hot air.

Further treatment preparing the trypsin solution was carried out following the procedure of Example 34, and further treatment preparing the resveratrol solution was carried out in a separate reactor following procedure of Example 38. The two solutions were mixed and agitated 5-7 min.

Then, 1.0 Kg of the obtained oxidized textile carrier was immersed in the resulting solution for activation. The carrier was then kept 2 hours at room temperature, and then dried by air.

Further treatment getting the microparticles was carried out following Example 1. Then 1 g of microparticles comprising trypsin and resveratrol were added to 99 g of cream and mixed during 5 min. The obtained cream contains 10 mg trypsin and 50 mg of resveratrol per 100 g of cream.

### Example 42

Bio-cream containing cellulose microparticles carriers at the size of 0.675-97.2 μm, partially oxidized comprising mexidole was carried out following the procedure of Example 1 except that 185 g of periodate nitrate were added to the reactor which contained 3.3 L of water and stirred. Subsequently the obtain dialdehyde cellulose contain 0.75 mg-equiv. aldehyde groups corresponding to 6% w/w per gram of a carrier.

A solution of mexidole was prepared in phosphate buffer solution following the procedure of Example 36 except that 50 g of mexidole was added to 3.3 L of phosphate buffer solution at pH 5.0 and the resulting mixture was stirred until completely dissolved.

1.0 Kg of the obtained oxidized textile carrier was immersed in the resulting solution for activation. The carrier was then kept 2 hours at room temperature, and then dried by air.

Further treatment was carried out following the procedure of Example 36. Subsequently The resulted cream containing 50 mg of mexidole per 100 g of cream.

### Example 43

Bio-cream containing cellulose microparticles carriers at the size of 0.675-97.2 μm, partially oxidized comprising trypsin and curcumin and resveratrol was carried out following the procedure of Example 1 except that 185 g of periodate nitrate were added to the reactor which contained 8 L of water and stirred. Subsequently the obtain dialdehyde cellulose contain 0.75 mg-equiv. aldehyde groups corresponding to 6% w/w per gram of a carrier.

A solution of trypsin was prepared in phosphate buffer solution of pH 5.5 following the procedure of Example 34 except that 10 g of trypsin was added to 3.3 L of phosphate buffer solution at pH 5.5, and the resulting mixture was stirred until completely dissolved.

A solution of resveratrol was prepared in an additional reactor contains 3.3 L phosphate buffer solution at pH 5.0 made by following the procedure of Example 34 except that 50 g resveratrol was added to the buffer solution and mixed. To prepar curcumin solution, 50 g of curcumin was added into a 3rd reactor contains 3.3 L phosphate buffer solution at pH 5.0 and the resulting mixture was stirred until completely dissolved. The 3 solutions were combined in a ratio of 1/1/1 and agitated during 5-7 min.

1.0 Kg of the obtained oxidized textile carrier was immersed in the resulting solution for activation. The carrier was then kept 2 hours at room temperature, and then dried by air.

Further treatment was carried out following the procedure of Example 36. Subsequently The resulted cream containing 10 mg of trypsin, 50 mg resveratrol and 50 mg of curcumin per 100 g of cream.

### Example 44

Bio-cream containing trypsin and chlorhexidine was made as follows: medical cotton gauze was activated by oxidation with sodium periodate to prepare dialdehyde cellulose as follows: 3.3 L of water were poured into a reactor, mixed with a stirrer and 6.0 g of iodic acid were added thereto. The same amount of water was poured into another reactor and 1.90 g of sodium hydroxide were added to the water using a stirrer. Both solutions were agitated until the crystals of the acid and sodium hydroxide were dissolved within 5-15 minutes. The solutions were then poured into a reactor, agitated for 3-6 minutes to give a sodium periodate solution have a pH of 5.5

1 Kg of medical cotton gauze was placed into the resulting solution and held (activated) at room temperature for 14 hours in the dark. After activating, the cloth was squeezed out, washed with 4×10 L of water and squeezed again. As a result of activation, the cloth acquires aldehyde group containing 0.04 mg-equiv. per gram of the textile carrier.

A solution of trypsin is prepared in phosphate buffer having a pH of 5.5 as follows: 10 g of trypsin and 330 ml of chlorhexidine gluconate of 20% were added to 3.3 L of the prepared phosphate buffer solution, stirring until it was completely dissolved. The activated textile carrier was reacted by impregnation. The resulting material was held in air for 2 hours and dried.

Further treatment was carried out following the procedure of Example 36.

### Example 45

Bio-cream containing chlorhexidine was made as follows: a knitted fabric from caproamide fibers was activated as follows: 40 L of 3M hydrochloric was poured into a reactor and 1.0 kg of knitted fabric from polycaproarnide fibers was placed therein. The temperature was adjusted to 60° C. and the fabric was kept at this temperature for 4 hours. Upon completion of hydrolysis, excess hydrochloric and was cooled and discarded. The knitted fabric was washed with water until no hydrochloric acid was observed in washings.

38.4 L of 5% a glutaric aldehyde was poured into the reactor, and 1.0 kg of the knitted fabric subjected to hydrolysis was placed therein. The temperature of glutaric aldehyde solution was adjusted to 50° C. and maintained for 4 hours. Then the glutaric aldehyde solution was cooled and poured out and after that the fabric was washed with water until there was no smell of glutaric acid. As a result of activation, the knitted fabric acquires aldehyde groups containing 0.05 mg-equiv., per gram of textile fabric. 0.33 of trypsin was added to 3.3 L of the prepared phosphate buffer solution, stirring until it was completely dissolved.

330 ml of chlorhexidine gluconate of 20% was added to 3.3 L of the prepared phosphate buffer solution, stirring until it was completely dissolved. The prepared solutions were mix at the ratio 1/1 and agitated during 5-7 min. The activated textile carrier was reacted by impregnation. The resulting material was held in air for 2 hours and dried.

Further treatment was carried out following the procedure of Example 36.

### Example 46

Bio-cream containing trypsin and insulin was made as follows: a cotton fabric was activated following the procedure of Example 34, except that 40 L of 3M of hydrochloric acid and 32 L of 5% a glutaric aldehyde were used. A resulting knitted fabric acquired aldehyde groups containing 0.05 mg-equiv. per gran1 of textile fabric.

Solutions of trypsin and insulin were prepared following the procedure of Example 11. The prepared solutions are stirred in equal volume ratio, and the textile carrier is activated with the obtained mixture by impregnation. Then the resulting material is held in air for 2 hours and dried.

Further treatment was carried out following the procedure of Example 36.

### Example 47

Bio-cream containing trypsin and mexidole was made as follows: medical cotton gauze was activated to obtain dialdehyde cellulose following the procedure of Example 36, except that 9.0 g of iodic acid and 1.5 g of sodium hydroxide were used. The resulting aldehyde cellulose contains 0.06 mg-equiv. of aldehyde groups per 1 gram of a carrier.

The trypsin solution was prepared in a reactor containing 3.3 L phosphate buffer solution having pH 5.0 by adding 10 g of trypsin to the buffer solution following the procedure of Example 34. The mexidole solution was prepared in a separate reactor containing 3.3 L phosphate buffer of pH 5.0 by following the procedure of Example 34, except that 50 g of mexidole was added to the buffer solution and stirred.

The prepared solutions are combined in equal volume ratio 1/1, stirred for 5-7 min. The textile carrier is activated with the obtained mixture by impregnation. The resulting material is then kept in air for 2 hours and dried.

Further treatment was carried out following the procedure of Example 36. The resulted cream contains 10 mg trypsin and 50 mg mexidole per 100 g of cream.

### Example 48

Bio-cream containing trypsin and lysozyme was made as follows: a knitted fabric from caproamide fibers was activated following the procedure of Example 42, except that 32 L of 3M of hydrochloric acid and 25.6 L of a glutaric aldehyde were used. A resulting knitted fabric acquires aldehyde groups containing 0.04 mg-equiv. per gram of textile fabric.

Then trypsin solution was prepared in following the procedure of Example 1 by adding 10 g of trypsin into the solution, except that the reactor contains 3.3 L buffer solution at pH 5.0

Lysozyme solution was prepared in another reactor in a phosphate buffer solution at pH 5.0 by adding 0.5 g in following the procedure of Example 16.

The prepared solutions were mixed and agitated in equal volume ratio during 5-7 min. 1 kg textile carrier was activated with the obtained mixture by impregnation. The resulting material is then kept in air for 2 hours and dried.

Further treatment was carried out following the procedure of Example 36. The resulted cream contains 10 mg trypsin and 0.5 mg lysozyme per 100 g cream.

### Example 49

Bio-active serum containing cellulose microparticles carriers on the order of 0.675-97.2 μm (in the form of dry powder) partially oxidized, comprising a bioactive substance—trypsin—was made as follow: medical cotton gauze was activated to obtain dialdehyde cellulose 30 g of periodate nitrate were added to the reactor which contained 8 L of water and stirred. The periodate nitrate was dissolved during 5-10 minutes. The resulted solution has a pH of 5.0.

1 Kg of medical cotton gauze was placed into the resulting solution and held (activated) at room temperature for 14 hours in the dark. After activating, the cloth was squeezed out, washed with 4×10 L of water, squeezed again and dried.

As a result of activation, the cotton cloth acquires 1.0% w/w of aldehyde groups corresponding to 0.125 mg-equiv. per gram of the textile carrier.

Further treatment was carried out following the procedure of Example 3 except that to prepare a trypsin solution, 10 g of trypsin were added to 3.3 L contains phosphate buffer solution having a pH of 5.5. Consequently, the resulting carriers all along the way contain 1.0% w/w of trypsin per Carrier.

Then, by a Vortex milling machine 1 kg of the obtained cloth was grinded—milled to microparticles carries, in the form of dry powder, on the order of 0.675-97.2 μm following Example 1. The properties of said treated cloth were entirely preserved so the DAC microparticles carriers contains 10 mg of trypsin per 1 g of microparticles carriers.

One (1) g. of said bio-active microparticles carriers was add to 99 g of serun (any serum) mixed 1-2 min by a mixer. The obtained serum contains 10 mg trypsin per 100 g cream.

### Example 50

A bio-serum comprising trypsin was made following the procedure of Example 36 except that to the reactor which contained 8 L of water 90 g of periodate nitrate were added thereto and stirred. The periodate nitrate was dissolved within 5-10 minutes.

1 Kg of medical cotton gauze was placed into the resulting solution and held (activated) at room temperature for 14 hours in the dark. After activating, the cloth was squeezed out, washed with 4×10 L of water, squeezed again and dried.

As a result of activation, the cotton cloth acquires 3.0% of dialdehyde groups corresponding to 0.375 mg-equiv. per gram of the textile carrier.

Further treatment was carried out following the procedure of Example 36 except that 30 g of trypsin were added to bio-active—trypsin solution. The obtained serum contains 30 mg of trypsin per 100 g of cream.

### Example 51

A bio-serum comprising trypsin was made following the procedure of Example 36 except that to the reactor which contained 8 L of water 372 g of periodate nitrate were added and as a result of the activation the cotton cloth acquires 12% of dialdehyde groups corresponding to 1.5 mg-equiv. per one gram of textile carrier.

Further treatment was carried out following the procedure of Example 34 except that 100 g of trypsin were added to the bio-active—trypsin solution. Subsequently the obtained serum contains 100 mg of trypsin per 100 g of serum.

### Example 52

A bio-serum comprising superoxide dismutase (SOD) was made following the procedure of Example 34 except that to the reactor which contained 8 L of water 185 g of periodate nitrate were added and as a result of the activation the cotton cloth acquires 6% of dialdehyde groups corresponding to 0.75 mg-equiv. per one gram of textile carrier

Further treatment was carried out following the procedure of Example—3 except that 50 g of SOD were added to the phosphate buffer solution.

Further treatment was carried out following Example 36. The obtained serum contains 50 mg of SOD per 100 g of serum.

### Example 53

Bio-serum containing cellulose microparticles carriers at the size of 0.675-97.2 μm partially oxidized comprising resveratrol was carried out following the procedure of Example 34, except that 185 g of periodate nitrate were added to the reactor which contained 8 L of water and stirred. Subsequently the obtain dialdehyde cellulose contain 0.75 mg-equiv. aldehyde groups corresponding to 6% w/w per gram of a carrier.

A solution of resveratrol was prepared in phosphate buffer solution following the procedure of Example 34 except that 50 g of resveratrol was added to 3.3 L phosphate buffer solution at pH 5.5, and the resulting mixture was stirred until completely dissolved.

1.0 Kg of the obtained oxidized textile carrier was immersed in the resulting solution for activation. The carrier was then kept 2 hours at room temperature, and then dried by air.

The further treatment was carried out following the procedure of Example 36. Subsequently The resulted serum contains 50 mg of resveratrol per 100 g of serum.

### Example 54

Bio-serum containing trypsin and resveratrol was made as follows: Medical cotton gauze was activated to obtain oxidized cellulose cloth containing 0.125 mg-equiv. of aldehyde groups corresponding to 1% (w/w) per gram of carrier, by following procedure Example 3. except that 50 g of 3-hydroxy-6-methyl-2-ethypyridine succinate (mexidole) was added to 3.3 L phosphate buffer solution at pH 5.5, and the resulting mixture was stirred to complete dissolution. 1 kg of the obtained oxidized cloth was placed into the mexidole solution for 2 hours at room temperature and then squeezed by a wringer between two rollers at a pressure of 25 Kg per 1 sq. cm and, dried by utilizing hot oven 90° C., or by forced hot air.

Further treatment preparing the trypsin solution was carried out following the procedure of Example 34, and further treatment preparing the resveratrol solution was carried out in a separate reactor following procedure of Example 38. The two solutions were mixed and agitated 5-7 min.

Then, 1.0 Kg of the obtained oxidized textile carrier was immersed in the resulting solution for activation. The carrier was then kept 2 hours at room temperature, and then dried by air.

Further treatment getting the microparticles was carried out following Example 1. Then 1 g of microparticles comprising trypsin and resveratrol were added to 99 g of serum and mixed during 5 min. The obtained serum contains 10 mg trypsin and 50 mg of resveratrol per 100 g of serum.

### Example 55

Bio-serum containing cellulose microparticles carriers at the size of 0.675-97.2 μm, partially oxidized comprising mexidole was carried out following the procedure of Example 3 except that 185 g of periodate nitrate were added to the reactor which contained 3.3 L of water and stirred. Subsequently the obtain dialdehyde cellulose contain 0.75 mg-equiv. aldehyde groups corresponding to 6% w/w per gram of a carrier.

A solution of mexidole was prepared in phosphate buffer solution following the procedure of Example 34 except that 50 g of mexidole was added to 3.3 L of phosphate buffer solution at pH 5.0 and the resulting mixture was stirred until completely dissolved.

1.0 Kg of the obtained oxidized textile carrier was immersed in the resulting solution for activation. The carrier was then kept 2 hours at room temperature, and then dried by air.

Further treatment was carried out following the procedure of Example 36. Subsequently The resulted serum containing 50 mg of mexidole per 100 g of serum.

### Example 54

Bio-serum containing cellulose microparticles carriers at the size of 0.675-97.2 μm, partially oxidized comprising trypsin and curcumin and resveratrol was carried out following the procedure of Example 1 except that 185 g of periodate nitrate were added to the reactor which contained 8 L of water and stirred. Subsequently the obtain dialdehyde cellulose contain 0.75 mg-equiv. aldehyde groups corresponding to 6% w/w per gram of a carrier.

A solution of trypsin was prepared in phosphate buffer solution of pH 5.5 following the procedure of Example 36 except that 10 g of trypsin was added to 3.3 L of phosphate buffer solution at pH 5.5, and the resulting mixture was stirred until completely dissolved.

A solution of resveratrol was prepared in an additional reactor contains 3.3 L phosphate buffer solution at pH 5.0 made by following the procedure of Example 36 except that 50 g resveratrol was added to the buffer solution and mixed. To prepare curcumin solution, 50 g of curcumin was added into a 3rd reactor contains 3.3 L phosphate buffer solution at pH 5.0 and the resulting mixture was stirred until completely dissolved. The 3 solutions were combined in a ratio of 1/1/1 and agitated during 5-7 min.

1.0 Kg of the obtained oxidized textile carrier was immersed in the resulting solution for activation. The carrier was then kept 2 hours at room temperature, and then dried by air.

Further treatment was carried out following the procedure of Example 36. Subsequently The resulted serum containing 10 mg of trypsin, 50 mg resveratrol and 50 mg of curcumin per 100 g of cream.

### Example 57

Bio-serum containing trypsin and chlorhexidine was made as follows: medical cotton gauze was activated by oxidation with sodium periodate to prepare dialdehyde cellulose as follows: 3.3 L of water were poured into a reactor, mixed with a stirrer and 6.0 g of iodic acid were added thereto. The same amount of water was poured into another reactor and 1.90 g of sodium hydroxide were added to the water using a stirrer. Both solutions were agitated until the crystals of the acid and sodium hydroxide were dissolved within 5-15 minutes. The solutions were then poured into a reactor, agitated for 3-6 minutes to give a sodium periodate solution have a pH of 5.5

1 Kg of medical cotton gauze was placed into the resulting solution and held (activated) at room temperature for 14 hours in the dark. After activating, the cloth was squeezed out, washed with 4×10 L of water and squeezed again. As a result of activation, the cloth acquires aldehyde group containing 0.04 mg-equiv. per gram of the textile carrier.

A solution of trypsin is prepared in phosphate buffer having a pH of 5.5 as follows: 10 g of trypsin and 330 ml of chlorhexidine gluconate of 20% were added to 3.3 L of the prepared phosphate buffer solution, stirring until it was completely dissolved. The activated textile carrier was reacted by impregnation. The resulting material was held in air for 2 hours and dried.

Further treatment was carried out following the procedure of Example 36.

### Example 58

Bio-serum containing chlorhexidine was made as follows: a knitted fabric from caproamide fibers was activated as follows: 40 L of 3M hydrochloric was poured into a reactor and 1.0 kg of knitted fabric from polycaproarnide fibers was placed therein. The temperature was adjusted to 60° C. and the fabric was kept at this temperature for 4 hours. Upon completion of hydrolysis, excess hydrochloric and was cooled and discarded. The knitted fabric was washed with water until no hydrochloric acid was observed in washings.

38.4 L of 5% a glutaric aldehyde was poured into the reactor, and 1.0 kg of the knitted fabric subjected to hydrolysis was placed therein. The temperature of glutaric aldehyde solution was adjusted to 50° C. and maintained for 4 hours. Then the glutaric aldehyde solution was cooled and poured out and after that the fabric was washed with water until there was no smell of glutaric acid. As a result of activation, the knitted fabric acquires aldehyde groups containing 0.05 mg-equiv., per gram of textile fabric. 0.33 of trypsin was added to 3.3 L of the prepared phosphate buffer solution, stirring until it was completely dissolved.

330 ml of chlorhexidine gluconate of 20% was added to 3.3 L of the prepared phosphate buffer solution, stirring until it was completely dissolved. The prepared solutions were mix at the ratio 1/1 and agitated during 5-7 min. The activated textile carrier was reacted by impregnation. The resulting material was held in air for 2 hours and dried.

Further treatment was carried out following the procedure of Example 36.

### Example 59

Bio-serum containing trypsin and insulin was made as follows: a cotton fabric was activated following the procedure of Example 34, except that 40 L of 3M of hydrochloric acid and 32 L of 5% a glutaric aldehyde were used. A resulting knitted fabric acquired aldehyde groups containing 0.05 mg-equiv. per gran1 of textile fabric.

Solutions of trypsin and insulin were prepared following the procedure of Example 11. The prepared solutions are stirred in equal volume ratio, and the textile carrier is activated with the obtained mixture by impregnation. Then the resulting material is held in air for 2 hours and dried.

Further treatment was carried out following the procedure of Example 36.

### Example 60

Bio-serum containing trypsin and mexidole was made as follows: medical cotton gauze was activated to obtain dialdehyde cellulose following the procedure of Example 36, except that 9.0 g of iodic acid and 1.5 g of sodium hydroxide were used. The resulting aldehyde cellulose contains 0.06 mg-equiv. of aldehyde groups per 1 gram of a carrier.

The trypsin solution was prepared in a reactor containing 3.3 L phosphate buffer solution having pH 5.0 by adding 10 g of trypsin to the buffer solution following the procedure of Example 36. The mexidole solution was prepared in a separate reactor containing 3.3 L phosphate buffer of pH 5.0 by following the procedure of Example 36, except that 50 g of mexidole was added to the buffer solution and stirred.

The prepared solutions are combined in equal volume ratio 1/1, stirred for 5-7 min. The textile carrier is activated with the obtained mixture by impregnation. The resulting material is then kept in air for 2 hours and dried.

Further treatment was carried out following the procedure of Example 36. The resulted serum contains 10 mg trypsin and 50 mg mexidole per 100 g of serum.

### Example 61

Bio-serum containing trypsin and lysozyme was made as follows: a knitted fabric from caproamide fibers was activated following the procedure of Example 42, except that 32 L of 3M of hydrochloric acid and 25.6 L of a glutaric aldehyde were used. A resulting knitted fabric acquires aldehyde groups containing 0.04 mg-equiv. per gram of textile fabric.

Then trypsin solution was prepared in following the procedure of Example 1 by adding 10 g of trypsin into the solution, except that the reactor contains 3.3 L buffer solution at pH 5.0

Lysozyme solution was prepared in another reactor in a phosphate buffer solution at pH 5.0 by adding 0.5 g in following the procedure of Example 16.

The prepared solutions were mixed and agitated in equal volume ratio during 5-7 min. 1 kg textile carrier was activated with the obtained mixture by impregnation. The resulting material is then kept in air for 2 hours and dried.

Further treatment was carried out following the procedure of Example 36. The resulted serum contains 10 mg trypsin and 0.5 mg lysozyme per 100 g serum.

1. A composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group.

2. The composition of claim 1, wherein said composition is for oral administration.

3. The composition of claim 2, wherein said composition is a tablet, capsule, powder, food product, syrup or liquid.

4. The composition of claim 1, wherein said at least one protein or polypeptide or therapeutic compound comprises trypsin, insulin, curcumin or reservatrol, lysozyme or a combination thereof, or mexidole, or chlorhexidine or a cannabinoid or a superoxide dismutase or glatiramer acetate (copaxsone).

5. (canceled)

6. (canceled)

7. (canceled)

8. (canceled)

9. The composition of claim 1, wherein said composition is for topical administration.

10. The composition of claim 9, wherein said composition is in the form of a cream, mousse, serum, shampoo, conditioner or makeup.

11. (canceled)

12. The composition of claim 1, wherein said at least one protein or polypeptide or therapeutic compound is trypsin at a dosage of from about 10 mcg to 50 mcg per 99 g of cream or serum.

13. (canceled)

14. (canceled)

15. (canceled)

16. (canceled)

17. The composition of claim 1, wherein said ground or milled cotton fibers comprising oxidized cellulose microparticles carriers of between about 10-15 μm in diameter and of between about 0.6-98 μm in length.

18. The composition of claim 1, wherein said ground or milled cotton fibers comprising oxidized cellulose microparticles carriers of between about 10.2-15.2 μm in diameter and of between about 0.675-97.2 μm in length.

19. A method for preparing a composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group;
Said method comprising the step of:
activating cotton fibers containing material comprising cellulose via application of a solution containing aldehyde functional groups to achieve oxidized cellulose components of said fibers thereof;
pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier;
applying a solution of one protein or polypeptide or therapeutic compound to said activated textile; and
drying said activated textile; and
Grinding or milling said activated textile to obtain oxidized cellulose microparticles carriers about 7-20 μm in diameter and of between about 0.5-100 μm in length;
Wherein a minority of about 20% of said protein or polypeptide or therapeutic compound is covalently bound and a majority of about 80% of said protein or polypeptide or therapeutic compound is non-covalently associated with said cellulose microparticles carriers.

20. A method for improving stability of a protein, polypeptide or therapeutic compound in a composition for delivery to a mucosal surface of a subject, said composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group;
Said method comprising the step of:
activating cotton fibers containing material comprising cellulose via application of a solution containing aldehyde functional groups to achieve oxidized cellulose components of said fibers thereof;
pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier;
applying a solution of one protein or polypeptide or therapeutic compound to said activated textile; and
drying said activated textile; and
Grinding or milling said activated textile to obtain oxidized cellulose microparticles carriers about 7-20 μm in diameter and of between about 0.5-100 μm in length;
Wherein a minority of about 20% of said protein or polypeptide or therapeutic compound is covalently bound and a majority of about 80% of said protein or polypeptide or therapeutic compound is non-covalently associated with said cellulose microparticles carriers; and
Whereby said protein or polypeptide or therapeutic compound agent stability in said composition is improved as compared to existing compositions containing said agent.

21. A method for improving a release profile of a protein, polypeptide or therapeutic compound in a composition for delivery to a mucosal surface of a subject, said composition comprising:
ground or milled cotton fibers comprising oxidized cellulose microparticles carriers of between about 7-20 μm in diameter and of between about 0.5-100 μm in length, wherein said oxidized cellulose microparticles carriers contain between about 0.208% w/w considerably 0.026 mg equiv. to 12.0% w/w considerably 1.5 mg-equiv. of aldehyde functional groups per gram of carrier, and wherein said carrier may range from about 100 mg to about 500 mg in weight; and
at least one protein or polypeptide or therapeutic compound, wherein said protein or polypeptide or therapeutic compound may be bound to said oxidized cellulose microparticles carrier through said aldehyde functional group;
Said method comprising the step of:
activating cotton fibers containing material comprising cellulose via application of a solution containing aldehyde functional groups to achieve oxidized cellulose components of said fibers thereof;
pressing said activated textile to uniformly spread said functional groups into the volume of the carrier and on the surface thereof to form between about 0.208% w/w considerably 0.026 mg equiv. to 12.0% w/w considerably 1.5 mg-equiv. of aldehyde groups per gram of carrier;
applying a solution of one protein or polypeptide or therapeutic compound to said activated textile; and
drying said activated textile; and
Grinding or milling said activated textile to obtain oxidized cellulose microparticles carriers of between about 7-20 μm in diameter and of between about 0.5-100 μm in length;
Wherein a minority of about 20% of said protein or polypeptide or therapeutic compound is covalently bound and a majority of about 80% of said protein or polypeptide or therapeutic compound is non-covalently associated with said cellulose microparticles carriers; and
Whereby said protein or polypeptide or therapeutic compound agent is released in a sustained release model from said composition.

22. The method of claim 19, wherein said grinding or milling provides a powder, which may be incorporated in a tablet, capsule or sachet formulation.

23. The method of claim 19, wherein said grinding or milling provides a solid product that may be incorporated in a food product or dietary supplement.

24. The method of claim 19, wherein said grinding or milling provides a product for use in a topical composition.

25. The method of claim 24, wherein said topical composition is a cream, mousse, serum, shampoo, conditioner or makeup.

26. The method of claim 19, wherein said composition is for oral administration.

27. The method of claim 26, wherein said composition is a tablet, capsule, powder, food product, syrup or liquid.

28. The method of claim 19, wherein said at least one protein or polypeptide or therapeutic compound comprises trypsin, insulin, curcumin or reservatrol, lysozyme or a combination thereof, or mexidole, or chlorhexidine or a cannabinoid or a superoxide dismutase or glatiramer acetate (copaxsone).

29-42. (canceled)
